(19) United States
(12) Patent Application Publication
(10) Pub. No.: US 2010/0062012 A1
(43) Pub. Date: Mar. 11, 2010

(54) Negative Genetic Regulation of Cancer Cell Renewal in Synergy with Notch- or Numb-Specific Immunotherapy

(76) Inventors: Constantin G. Ioannides, Houston, TX (US); Raymund F. Eich, Houston, TX (US)

Correspondence Address: WILLIAMS, MORGAN & AMERSON, 10333 RICHMOND, SUITE 1100, HOUSTON, TX, 77042, US

(21) Appl. No.: 12/529,759

(22) PCT Filed: Feb. 8, 2008
(86) PCT No.: PCT/US08/01694

§ 371 Date: Nov. 3, 2009

Related U.S. Application Data
(60) Provisional application No. 60/904,994, filed on Mar. 5, 2007.
(60) Provisional application No. 60/961,046, filed on Jul. 18, 2007.
(60) Provisional application No. 60/959,971, filed on Jul. 18, 2007.
(60) Provisional application No. 60/959,946, filed on Jul. 18, 2007.

(51) Int. Cl. A61K 39/00; A61P 35/00

(52) U.S. Cl. 424/185.1

(57) ABSTRACT

We disclose a method of treating a cancer in a patient by immunizing the patient against a peptide derived from a protein selected from the group consisting of Notch 1, Notch2, Notch3, and Notch4. We further disclose a composition containing a peptide as described above and a pharmaceutically-acceptable carrier. In addition, we disclose a method of treating a cancer in a patient by immunizing the patient against a peptide derived from a protein selected from the group consisting of Numb1, Numb2, Numb3, and Numb4. We also disclose a composition containing a peptide as described above and a pharmaceutically-acceptable carrier. Further, we disclose a method of treating a cancer in a patient by administering to the patient a composition comprising an antibody against a peptide derived from a protein selected from the group consisting of Notch 1, Notch2, Notch3, Notch4, Numb1, Numb2, Numb3, and Numb4.

### BACKGROUND OF THE INVENTION

The present invention relates generally to the field of cancer therapy. More particularly, it concerns compositions and methods for treating cancers characterized by upregulation, overexpression, or disinhibition of Notch, Numb, or both.

Notch is a plasma membrane receptor involved in the control of cell fate specification and in the maintenance of the balance between proliferation and differentiation in many cell lineages (1, 2). Notch signaling is important in regulating numerous physiological processes, and disruption of Notch has been implicated in a variety of hematological and solid cancers.

The best-studied example is the link between mutations of Notch1 and T-cell acute lymphoblastic leukemia and lymphoma (T-ALL). In a subset of T-ALL tumor cells, a (7, 9) chromosomal translocation fuses the 3′ portion of Notch1 to the T-cell receptor Jβ locus.

This results in a truncated Notch1 protein, which is constitutively active and aberrantly expressed (3). In addition, activating mutations in Notch1 independent of the (7, 9) translocation have been found in more than 50% of human T-ALL cases (4).

Abnormal Notch signaling has also been reported in solid tumors, including cancers of the breast, pancreas, prostate, liver, stomach and colon cancer, although without evidence of genetic lesions (5-7). Notch may play either an oncogenic or a tumor-suppressive role, depending on the cancer type, other signaling pathways present and the identity of Notch receptor activated.

However, in a large majority of cases including breast cancer, Notch signaling promotes tumor growth (8). One mechanism for the oncogenic role of Notch may derive from its ability to prevent differentiation and maintain the stem cell phenotype. Stem cells and tumor cells share common characteristics, such as unlimited proliferation and undifferentiation. Further, self-renewal in stem cells and tumor cells are regulated by similar pathways, including sonic hedgehog, Wnt and Notch. It is possible that tumor cells may derive from normal stem cells or that cancers may harbor “cancer stem cells” that are resistant to treatment (9).

During asymmetric cell division in embryogenesis, the activity of Notch is biologically antagonized by the cell fate determinant Numb (11, 12). The asymmetric cell division consists in division of a stem cell in a differentiated and in a non-differentiated daughter. Numb is also expressed in many adult mammalian cells (13). Adult cells divide symmetrically, and Numb is symmetrically partitioned where at mitosis. The symmetric partitions suggest that either Numb is inactive or has additional functions. The Numb/Notch antagonism is relevant to control of the division of the normal mammary parenchyma. The normal breast parenchyma invariably expresses intense and homogeneous Numb staining. In contrast, tumors display marked heterogeneity and in many cases complete absence of Numb immunoreactivity (14, 15).

Based on this and additional information, it is believed that subversion (by blocking or inhibition) of the Numb-mediated regulation of Notch plays a causative role in naturally occurring breast cancers. 80% of breast tumors show Numb immunoreactivity in 50% of the tumor cells. Thus, almost one half of all breast tumors have reduced levels of Numb. A strong inverse correlation was found between Numb expression levels and tumor grade and Ki67 labeling index, which are known indicators of aggressive disease (14). The low Numb levels were reported to be restored to high levels by treatment with proteasome inhibitors such as MG132 (14). Reduction of Numb levels in breast tumors studied did not appear to be the consequence of a generally increased proteasomal activity, as the basal levels of other cellular proteins also regulated by proteasomal degradation, were not affected under the same experimental conditions, although this matter requires further investigation.

### SUMMARY OF THE INVENTION

In one embodiment, the present invention relates to a method of treating a cancer in a patient by immunizing the patient against a peptide derived from a protein selected from the group consisting of Notch1, Notch2, Notch3, and Notch4.

In one embodiment, the present invention relates to a composition containing a peptide as described above and a pharmaceutically-acceptable carrier.

In one embodiment, the present invention relates to a method of treating a cancer in a patient by immunizing the patient against a peptide derived from a protein selected from the group consisting of Numb1, Numb2, Numb3, and Numb4.

In one embodiment, the present invention relates to a composition containing a peptide as described above and a pharmaceutically-acceptable carrier.

In one embodiment, the present invention relates to a method of treating a cancer in a patient by administering to the patient a composition comprising an antibody against a peptide derived from a protein selected from the group consisting of Notch1, Notch1, Notch3, Notch4, Numb1, Numb2, Numb3, and Numb4.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Molecular models of Notch1 C-terminal domain amino acids 1902-2143 (A, B) and Numb1 phosphotyrosine-binding domain (PTB) (C, D). (B, D) show the charges of these molecules, red indicate positive charge, blue indicate negative charge. The positions of Notch1-1947, Notch1-2112, and Numb1-87 peptides are shown in (A, C).

FIG. 2. Expression of Notch1 on breast MCF7 and ovarian SK-OV-3 tumor cell lines. (A, B, C) cells stained with isotype control antibody. (D, E, F) cells stained with antibody against Notch1. MCF7 (A, D), SK-OY-3 (B, E), and SK-LMS-1 leiomyosarcoma (C, F).

FIG. 3. Kinetics of proliferation of TAL-1. Freshly isolated TAL-1 were cultured with 150 IU/ml IL-S. Most cells died in low concentration of IL-2 in the first 8 days. Surviving cells increased in numbers afterwards.

FIG. 4. (A) TAL-1 stained with HLA-A2-lgG dimer not pulsed with peptide (dNP) was used as a negative dimer control. (B) TAL-1 stained with Notch 1-2112 peptide HLA-A2-IgG dimmer (dNotchl-2112). (C) TAL-1 stained Numb1-87-HLA-A2 peptide dimer (dNumbl-87). Note a 3.3-fold increase the numbers of TCR^{hi} Per^{hi} cells compared with B. (D) TAL-1 stained with AES1-HLA-A2-IgG peptide dimer. (E-H) TAL-1 stained with antibody against Perforin. (G) Numb1-87-TCR⁺ cells have the highest amount of Perforin.

FIG. 5. (A-D) Analysis of to all gated in TAL-2. (A) TAL-2 stained with HLA-A2-IgG dimer not pulsed with peptide (dNP) was used as a negative dimer control. (B) TAL-2 stained with Notch1-1947 peptide HLA-A2-IgG dimmer (dNotch1-1947), (C) TAL-2 stained with Notch1-2112-HLA-A2-IgG dimer (dNotch2112), (D) TAL-2 stained with Numb1-87-J-ILA-A2-lgG peptide dimer (dNumb 1-87). (E-H) Analysis of large-size lymphocytes TAL-2. (E) dNP, (F) Notch1-1947, (G) Notch1-2112, (H) Numb1-87 increase 3-fold the numbers of TCR1a.

FIG. 6. Expression of ESA, CD44, and CD24 on cancer cell lines. Cells cultured with or without gemcitabine were gated for ESA. CD44 and CD24 were analyzed. ESA⁺CD44^{hi} CD24^{low/-} population was relative high and there was no different change of expression of those markers by GEM-treatment on PANC-1 and AsPC-1. ESA⁺ CD44^{hi} CD24^{low/-} cells of BR-C line MCF7 was known as CSt-Cs, and its population increased with GEM-treatment. (A) PANC-1; (B) MCF7; (C) SKOV-3; (D) MIA PaCa-2; (E) MCF7.

FIG. 7. (A) The number of cells expressing the NKG2D ligands MICA and MICB increased in Gem^{Res} and FU^{Res} MIA PaCa-2. The MIC-A/B⁺ cells did not increase in number in PTX^{Res} cells. (B) Similar results with drug-resistant positive control MCF-7 cells. White peak represents -? ESA+ cells ? Black peaks show the MIC-A/B⁺ cells. The % MICA-A/B⁺ cells is shown underlined. The increase in numbers of MICA-A/B⁺ cells was not paralleled by an increase in the MIC-A/B density per drug resistant cell.

FIG. 8. Pancreatic cell lines contain CD133⁺ cells, whose number increased in drug resistant populations. Populations which shared expression of CSC markers (CD44⁺CD24^{low}, CD44⁺ CD133⁺, and CD24^{low} CD133⁺) increased after treatment with gemcitabine. (*) substantial increase more than 2-fold. (white) untreated cells, (black) drug resistant cells. MCF-7 and SKOV3 were used as positive controls for CD44, CD24, and ESA markers. Selection of drug resistant cells and quantification of cells of CSC phenotype was made as described in Materials and Methods. (A) The ESA⁺ CD44^{hi} CD24^{low} and CD133⁺ populations increased in the GEM^{Res} population by 3-5 fold compared with the entire population in Mia-PaCa-2, PANC-1, MCF7 and SKOV3, but not in AsPC-1. (B) A large number of DLL4-expanded cells were of CD44^{low} CD24^{lo} and CD24^{hi} phenotype. (C) Comparable results were observed for the CD44⁺ CD133⁺ phenotype. (D) Comparable results were observed for the CD24^{low} CD133⁺ phenotype.

FIG. 9. Cells surviving gemcitabine activate components of distinct survival pathways in Miapaca-2 and MCF-7. (A). NICD and Bcl-2 expression increased in Gem^{Res} MIA PaCa-2 compared with untreated (UT) Miapaca-2. (B) NECD expression increased and NICD expression decreased in MCF7 cells. One of two experiments is shown. (C, D) Diagram of increase in NECD expression in Gem^{Res} MCF-7 paralleled by decrease in the amounts of Numb^{S}, Numb^{L} and Bcl-2. Expression levels for each protein were normalized in relation to actin levels in the same sample separated on the same gel. Calculated used the formula: expression index (E.I.)=Optical density of a particular protein in a sample divided by the α-actin density of the protein in the same sample. Expression of Bcl-2 in MCF7 cells is shown from a membrane exposed for 10 min; Bcl-2 in MIA PaCa-2 is shown from the same membrane exposed for only 3 min. MCF7 had lower amount of BCl-2 than MIA PaCa-2. The E.I. for Bcl-2 in MCF7 cells was calculated from the optical density values at 3 min of exposure. Decreases in the amounts of proteins were considered substantial if the result of the division of the ratio {(NECD: Numb^{L})-GEM^{Res} to NECD:Numb^{L})-GEM^{Sens}} was higher or lower than 2; i.e. fold increase, or fold decrease. NE, NECD; NI, NICD; N-L, Numb^{L}, N-S, Numb^{S}.

FIG. 10. (A,B). Morphologic changes of Gem^{Res} MIAPaCa-2 compared with UT-Miapaca-2. UT-MIAPaCa-2 are round-shaped cells (A), but they transform into spindle-shaped cells with long tentacles after treatment with gemcitabine (B). (C). Low levels of expression of the MICA-A/B Ag per cell in Gem^{Res} MCF-7 cells. White peak, isotype control Ab; dark peak, MIC-A/B-specific Ab.

FIG. 11. (A). SKOV3.A2 cells present the Numb-1 (87-95) peptide to Numb-1 peptide activated PBMC. Substantially higher, by 2-fold IFN-g production by Numb-1-peptide activated PBMC than by Notch peptides activated PBMC. Note that at 48 h the amount of IFN g produced by the two Notch peptide activated cell lines and the non-specifically, IL-2-activated cell lines was low and similar. Only Notch peptide, 2112-2120, can be presented by HL-A2 after Notch digestion by proteasome. (the program paproc.de). (B). Western analysis of Notch and Numb protein expression in SKOV3. Numb S/L is expressed in significantly higher amount in SKOV3 than in MCF-7 but in similar amount in Miapaca-2. A part of Numb is phosphorylated. A small part of Numb was phosphorylated at the Ser²⁸³. A large part of Numb was phosphorylated at the Ser²⁶⁴. NECD was detected with mAbs-scc3275 (recognize the whole Notch molecule, and H131 (detected two polypeptides corresponding to NICD of 100 and 80 kDa respectively). (C) Presentation of Numb-1(87-95) peptide to Numb-1(87-95) peptide activated cells, is dependent on phosphorylation mediated by PKC-family members and at lesser extent by MAPK-kinases. PI3K does not appear to be involved in peptide presentation Treatement of SKOV3.A2 cells with the broad spectrum PKC kinase inhibitor, staurosporine, but not the PI3K inhibitor wortmanin (WT) abolished the IFN-g production by the indicator cell line. The MAPK-kinase SB20380 had a weaker inhibitory effect. The closed symbols indicate are 24 h measurements, the open symbols indicate 48 h measurements.

FIG. 12. MCF-7 were untreated (UT, Gem^{Sens}) or were cultured with Gemcitabine (300 nM Gem for 3 days, followed by 100 nM Gem for another 5 days, Gem^{Res}) Note increase in CD24^{neg/low} cells, but not in the MFI of CD24^{lo} and CD24^{hi} cells. This experiment was repeated in the same conditions and the data were confirmed. (data not shown).

FIG. 13. Cancer-stem-like cells (C-St-C) make cancer mass.

FIG. 14. Proposed mechanism of oncogenesis caused by overexpression of Aurora-A.

FIG. 15. A. Notch activated cancer cell proliferation. B. Numb functional repair following immunoselection.

### DESCRIPTION OF ILLUSTRATIVE EMBODIMENTS

In one embodiment, the present invention relates to a method of treating a cancer in a patient by immunizing the patient against a peptide derived from a protein selected from the group consisting of Notch1, Notch2, Notch3, and Notch4.

In one embodiment, the present invention relates to a method of treating a cancer in a patient by immunizing the patient against a peptide derived from a protein selected from the group consisting of Numb1, Numb2, Numb3, and Numb4.

In one embodiment, the present invention relates to a method of treating a cancer in a patient by administering to the patient a composition comprising an antibody against a peptide derived from a protein selected from the group consisting of Notch1, Notch2, Notch3, Notch4, Numb1, Numb2, Numb3, and Numb4.

There is a single Notch receptor and two ligands (Delta and Serrate) in Drosophila. In mammals, there are four receptors and five ligands. Notch 1-4 are homologues of Drosophila Notch; Delta-like-1, -3 and -4 (D111, D113, D114) are homologues of Delta; Jagged1 and Jagged2 (Jag1 and Jag2) are homologues of Serrate.

Each Notch receptor is synthesized as a full-length precursor protein consisting of extracellular, transmembrane and intracellular domains. Notch signaling is normally activated by ligand receptor binding between two neighboring cells. This interaction induces a conformational change in the receptor, exposing a cleavage site, S2, in its extracellular domain. After cleavage by the metalloprotease TNF-α converting enzyme (TACE) and/or Kuzbanian, Notch receptor undergoes intramembrane proteolysis at cleavage site S3. This cleavage, mediated by the γ-secretase complex, liberates the Notch intracellular domain (N-ICD), which then translocates into the nucleus to activate Notch target genes. Inhibiting γ-secretase function prevents the final cleavage of the Notch receptor, blocking Notch signal transduction. In the absence of N-ICD cleavage, transcription of Notch target genes is inhibited by a repressor complex mediated by the Suppressor of Hairless (re-combination-signal binding protein jκ (RBP-κ) homologue) in Drosophila.

Recent studies in Drosophila have suggested that Notch can signal independently of the canonical Suppressor of Hairless pathway. However, it is unclear if this is the case in vertebrates. Some early evidence from myogenic cell lines and the developing avian neural crest suggests that Notch signaling can occur in the presence of dominant negative Suppressor of Hairless, but additional characterization is needed to establish alternative downstream pathways in vertebrates (10).

The Notch1, Notch2, Notch3, and Notch4 of the present invention are mammalian proteins, and in one embodiment, are human proteins. In one embodiment, Notch1 has the sequence given as SEQ ID NO: 1. In one embodiment, Notch2 has the sequence given as SEQ ID NO:2. In one embodiment, Notch3 has the sequence given as SEQ ID NO:3. In one embodiment, Notch4 has the sequence given as SEQ ID NO:4.

Mammalian Numb has four splicing isoforms, Numb1 to Numb4, which are divided into two types (Numb^{L} and Numb^{S}) based on the presence or absence of a 49 amino acid insert (5 kDa) in the proline-rich region (PRR) in the C-terminus.

In one embodiment, Numb 1 has the sequence given as SEQ ID NO:5. In one embodiment, Numb2 has the sequence given as SEQ ID NO:6. In one embodiment, Numb3 has the sequence given as SEQ ID NO:7. In one embodiment, Numb4 has the sequence given as SEQ ID NO:8.

A “peptide” is used herein to refer to any oligomer containing from about five to about fifty amino acids. A peptide is “derived from” a protein if the peptide has at least about 95% identity with a subsequence of the amino acid sequence of the protein. In one embodiment, a peptide derived from a protein may have at least about 96% identity, such as about 97% identity, 98% identity, 99% identity, 99.5% identity, or 99.9% identity, with a subsequence of the amino acid sequence of the protein. As used herein, “derived from” neither states nor implies that the peptide must be produced by proteolysis of the protein. The peptide may be produced by proteolysis of the protein, by chemical synthesis in light of the amino acid sequence of the protein, by use of an organism expressing a nucleic acid sequence encoding the peptide, or by other techniques known in the art.

In one embodiment, the peptide is selected from the group consisting of DGVNTYNC (SEQ ID NO:9), RYSRSD (SEQ ID NO:11), LLEASAD (SEQ ID NO:18), LLDEYNLV (SEQ ID NO:21), MPALRPALLWALLALWLCCA (SEQ ID NO:22), NGGVCVDGVNTYNC (SEQ ID NO:25), DGVNTYNCRCPPQWTG (SEQ ID NO:30), RMNDGTTPLI (SEQ ID NO:32), and LKNGANR (SEQ ID NO:35).

In one embodiment, the peptide is selected from the group consisting of Notch1₂₇₄₋₂₈₂ (SEQ ID NO:10), Notch 1₁₉₃₈₋₁₉₄₃ (SEQ ID NO:11), Notch1₁₉₃₈₋₁₉₄₆ (SEQ ID NO:12), Notch1₁₉₃₈₋₁₉₄₇ (SEQ ID NO:13), Notch1₁₉₄₀₋₁₉₄₈ (SEQ ID NO:14), Notch1₁₉₄₀₋₁₉₄₉ (SEQ ID NO:15), Notch1₁₉₄₄₋₁₉₅₅ (SEQ ID NO:16), Notch1₁₉₄₇₋₁₉₅₅ (SEQ ID NO:17), Notch1₂₁₁₁-2120 (SEQ ID NO:19), Notch1₂₁₁₂₋₂₁₂₀ (SEQ ID NO:20), Notch1₂₁₁₃₋₂₁₂₀ (SEQ ID NO:21), Notch2₁₋₂₀ (SEQ ID NO:22), Notch2₇₋₁₅ (SEQ ID NO:24), Notch2₂₇₁₋₂₈₅ (SEQ ID NO:26), Notch2₂₇₁₋₂₈₆ (SEQ ID NO:27), Notch2₂₇₇₋₂₈₅ (SEQ ID NO:28), Notch2₂₇₇₋₂₈₆ (SEQ ID NO:29), Notch2₁₉₄₀₋₁₉₄₈ (SEQ ID NO:31), Notch2₁₉₄₀₋₁₉₄₉ (SEQ ID NO:32), Notch2₁₉₉₁₋₂₀₀₃ (SEQ ID NO:33), Notch2₁₉₉₅₋₂₀₀₃ (SEQ ID NO:34), and NOtch2₁₉₉₇₋₂₀₀₃ (SEQ ID NO:35).

In one embodiment, the peptide is selected from the group consisting of LWVSADGL (SEQ ID NO:37), CRDGTTRRWICHCFMAVKD (SEQ ID NO:38), RWICHCFMAVKD (SEQ ID NO:39), RWLEEVSKSVRA (SEQ ID NO:41), and VDDGRLASADRHTEV (SEQ ID NO:43).

In one embodiment, the peptide is selected from the group consisting of Numb1₈₇₋₉₅ (SEQ ID NO:36), Numb1₈₈₋₉₅ (SEQ ID NO:37), Numb1₁₃₁₋₁₄₉ (SEQ ID NO:38), Numb1₁₃₈₋₁₄₉ (SEQ ID NO:39), Numb1₁₃₉₋₁₄₇ (SEQ ID NO:40), Numb1₄₄₂₋₄₅₃ (SEQ ID NO:41), Numb1₄₄₃₋₄₅₁ (SEQ ID NO:42), Numb1₅₉₂₋₆₀₆ (SEQ ID NO:43), and Numb1₅₉₄₋₆₀₂ (SEQ ID NO:44).

The peptide may be a component of a composition which also contains a pharmaceutically-acceptable carrier, such as saline, among others known in the art. The peptide can be used to raise antibodies against it. Methods for production and purification of monoclonal antibodies or polyclonal antibodies (generically, “antibodies”) are known in the art. In one embodiment, the peptide is covalently linked with an HLA-A2 molecule in a manner such that antibodies can be raised against the peptide.

Once produced and purified, antibodies against the peptide can be administered directly to a patient to treat a cancer, or can be formed into a composition with other materials to yield a composition that can be administered to a patient to treat a cancer. In one embodiment, the antibody can be formed into a composition with a therapeutic molecule selected from the group consisting of anti-cancer drugs and radioisotopes. Exemplary anti-cancer drugs include, but are not limited to, paclitaxel (commercially available as Taxol, Bristol-Myers Squibb), doxorubicin (also known under the trade name Adriamycin), vincristine (known under the trade names Oncovin, Vincasar PES, and Vincrex), actinomycin D, altretamine, asparaginase, bleomycin, busulphan, capecitabine, carboplatin, carmustine, chlorambucil, cisplatin, cyclophosphamide, cytarabine, dacarbazine, daunorubicin, epirubicin, etoposide, fludarabine, fluorouracil, gemcitabine, hydroxyurea, idarubicin, ifosfamide, irinotecan, lomustine, melphalan, mercaptopurine, methotrexate, mitomycin, mitozantrone, oxaliplatin, procarbazine, steroids, streptozocin, taxotere, tamozolomide, thioguanine, thiotepa, tomudex, topotecan, treosulfan, UFT (uracil-tegufur), vinblastine, and vindesine, among others.

Radioisotopes known in the art of cancer radiotherapy include, but are not limited to, ¹²⁵I, ¹³¹I, ⁹⁰Y, ²²¹At, ²²⁵Ac, ²¹²Bi, ²¹³Bi, ⁹⁹Re, ¹⁶⁶Ho, ¹⁷⁷Lu, or ¹⁵³Sm, among others.

When the antibody is formed into a composition with the therapeutic molecule, in one embodiment, the therapeutic molecule is covalently linked to a constant region of a heavy chain of the antibody. In one embodiment, the therapeutic molecule can be covalently linked by, for example, (i) adding a sulfhydryl-containing (—SH) substituent to the therapeutic molecule; (ii) preparing the antibody with a sulfhydryl-containing substituent in a constant region of a heavy chain; and (iii) reacting the antibody and the therapeutic molecule across their sulfhydryl-containing substituents to form a —S—S— bond between the therapeutic molecule and the constant region of the heavy chain of the antibody.

In one embodiment, the composition comprising the peptide and the pharmaceutically-acceptable carrier may further comprise an adjuvant, such as an aluminum salt, QS21, MF59, or a virosome, among others known in the art.

The peptide can be administered to the patient with a pharmaceutically-acceptable carrier, if any, in any manner which the skilled artisan would expect to elicit formation of antibodies against the peptide. Methods of vaccination are well-known in the art. Administering the peptide can be used to treat any cancer characterized by upregulation, overexpression, or disinhibition of Notch or Numb. In one embodiment, the cancer is selected from the group consisting of T-cell acute lymphoblastic leukemia and lymphoma (T-ALL), breast cancer, ovarian cancer, pancreatic cancer, prostate cancer, liver cancer, stomach cancer, clear-cell renal cell carcinomas, and colon cancer.

“Immunizing against a peptide” and variations of this phrase are used to refer to the induction of the creation of one or more antibodies by the patient's immune system, wherein the antibody or antibodies recognize the peptide as an antigen. Though not to be bound by theory, by immunizing the patient against a peptide derived from a protein selected from the group consisting of Notch1, Notch2, Notch3, and Notch4, i.e., inducing the creation of an antibody or antibodies against the peptide, it is believed that at least some patients suffering from a cancer characterized by upregulation, overexpression, or disinhibition of Notch can be treated, that is, experience at least a partial reduction in tumor size or cancer cell count.

In one embodiment, the peptide is covalently linked with an HLA-A2 molecule prior to administration in a manner such that antibodies can be raised against the peptide after administration.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Abstract: Notch is a plasma membrane receptor involved in the control of cell fate specification and in the maintenance of the balance between proliferation and differentiation in many cell lineages. Disruption of Notch has been implicated in a variety of hematological and solid cancers. Numb is also expressed in many adult mammalian cells. Adult cells divide symmetrically, and Numb is symmetrically partitioned at mitosis. The Numb-mediated regulation of Notch is believed to play a causative role in naturally occurring breast cancers. Reduction of Numb levels in breast tumors is regulated by proteasomal degradation.

We reasoned that if the disregulated negative control of Notch by Numb protein is the consequence of Numb proteasomal degradation, then degradation of Numb can generate peptides which are transported presented by MHC-I molecules. Surprisingly, we found few candidate naturally processed peptides from Notch1, Notch2, and Numb1. CD8⁺ T cells expressing TCRs which specifically recognized peptides Notch1 (2112-2120) and Numb1 (87-95) were presented in the ascites of ovarian cancer patients. Many of these cells were differentiated and expressed high levels of Perforin.

The natural immunogenicity of Notch1 and particularly of Numb1 suggests a mechanism of immunosurveillance which is overcome during tumor progression. Immunotherapy with tumor antigens from Notch and Numb should be important for treatment of cancer patients.

Introduction: Notch is a plasma membrane receptor involved in the control of cell fate specification and in the maintenance of the balance between proliferation and differentiation in many cell lineages (1,2). Notch signaling is important in regulating numerous physiological processes, disruption of Notch has been implicated in a variety of hematological and solid cancers.

The best-studied example is the link between mutations of Notch1 and T-cell acute lymphoblastic leukemia and lymphoma (T-ALL). In a subset of T-ALL tumor cells, at (7; 9) chromosomal translocation fuses the 3′ portion of Notch1 to the T-cell receptor Jβ locus. This results in a truncated Notch1 protein, which is constitutively active and aberrantly expressed (3). In addition, activating mutations in Notch1 independent of the t (7; 9) translocation have been found in more than 50% of human T-ALL cases (4).

Abnormal Notch signaling has also been reported in solid tumors, including cancers of the breast, pancreas, prostate, liver, stomach and colon cancer, although without evidence of genetic lesions (5-7). Notch may play either an oncogenic or a tumor-suppressive role, depending on the cancer type, other signaling pathways present and the identity of Notch receptor activated.

However, in a large majority of cases including breast cancer, Notch signaling promotes tumor growth (8). One mechanism for the oncogenic role of Notch may derive from its ability to prevent differentiation and maintain the stem cell phenotype. Stem cells and tumor cells share common characteristics, such as unlimited proliferation and undifferentiation. Further, self-renewal in stem cells and tumor cells are regulated by similar pathways, including sonic hedgehog, Wnt and Notch. It is possible that tumor cells may derive from normal stem cells or that cancers may harbor “cancer stem cells” that are resistant to treatment (9).

There is a single Notch receptor and two ligands (Delta and Serrate) in Drosophila. In mammals, there are four receptors and five ligands, which are the focus of this review. Notch 1-4 are homologues of Drosophila Notch; Delta-like-1, -3 and -4 (D111, D113, D114) are homologues of Delta; Jagged1 and Jagged2 (Jag1 and Jag2) are homologues of Serrate.

Each Notch receptor is synthesized as a full-length precursor protein consisting of extracellular, transmembrane and intracellular domains. Notch signaling is normally activated by ligand receptor binding between two neighboring cells. This interaction induces a conformational change in the receptor, exposing a cleavage site, S2, in its extracellular domain. After cleavage by the metalloprotease TNF-α converting enzyme (TACE) and/or Kuzbanian, Notch receptor undergoes intramembrane proteolysis at cleavage site S3. This cleavage, mediated by the γ-secretase complex, liberates the Notch intracellular domain (N-ICD), which then translocates into the nucleus to activate Notch target genes. Inhibiting γ-secretase function prevents the final cleavage of the Notch receptor, blocking Notch signal transduction. In the absence of N-ICD cleavage, transcription of Notch target genes is inhibited by a repressor complex mediated by the Suppressor of Hairless (re-combination-signal binding protein jκ (RBP-jκ) homologue) in Drosophila.

Recent studies in Drosophila have suggested that Notch can signal independently of the canonical Suppressor of Hairless pathway. However, it is unclear if this is the case in vertebrates. Some early evidence from myogenic cell lines and the developing avian neural crest suggests that Notch signaling can occur in the presence of dominant negative Suppressor of Hairless, but additional characterization is needed to establish alternative downstream pathways in vertebrates (10).

During asymmetric cell division in embryogenesis, the activity of Notch is biologically antagonized by the cell fate determinant Numb (11,12). The asymmetric cell division consists in division of a stem cell in a differentiated and in a non-differentiated daughter. Numb is also expressed in many adult mammalian cells (13). Adult cells divide symmetrically, and Numb is symmetrically partitioned where at mitosis. The symmetric partitions suggest that either Numb is inactive or has additional functions. The Numb/Notch antagonism is relevant to control of the division of the normal mammary parenchyma. The normal breast parenchyma invariably expresses intense and homogeneous Numb staining. In contrast, tumors display marked heterogeneity and in many cases complete absence of Numb immunoreactivity (14,15).

Based on this and additional information, it is believed that subversion (by blocking or inhibition) of the Numb-mediated regulation of Notch plays a causative role in naturally occurring breast cancers. 80% of breast tumors show Numb immunoreactivity in 50% of the tumor cells. Thus, almost one half of all breast tumors have reduced levels of Numb. A strong inverse correlation was found between Numb expression levels and tumor grade and Ki67 labeling index, which are known indicators of aggressive disease (14). The low Numb levels were reported to be restored to high levels by treatment with proteasome inhibitors such as MG132 (14). Reduction of Numb levels in breast tumors studied did not appear to be the consequence of a generally increased proteasomal activity, as the basal levels of other cellular proteins also regulated by proteasomal degradation, were not affected under the same experimental conditions, although this matter requires further investigation.

We reasoned that if the disregulated negative control of Notch by Numb protein is the consequence of Numb proteasomal degradation, then degradation of Numb can generate peptides which are transported by Transporter associated with antigen processing (TAP) and presented by. MHC-I molecules. It is possible that T cells which recognize these MHC-I Numb peptide complexes are tolerized or eliminated in healthy individuals. Furthermore, if degradation of Notch is required for its signaling, then cytoplsmic degradation of the N-ICD should also generate Notch peptides. If some of the Notch fragments are degraded by the proteasome, they may be also presented by MHC-I molecules. If Notch and Numb peptides are not tolerogenic, then activated CD8⁺T cells bearing receptors for such peptides should be detected in vivo, in cancer patients. The current study was performed to address these hypotheses.

### Materials and Methods:

Identification of candidate MHC-I binding peptides with predictive algorithms. We used the following programs to identify peptides which can bind HLA-A, B, C and HLA-DR molecules: (1) BIMAS (Informatics and Molecular Analysis Section.) to predict peptides binding to HLA-A, B, C. (http://bimas.cit.nih.gov/molbio/hla_bind) (16); (2) PAPROC (Prediction Algorithm for Proteasomal Cleavages). PAPROC is a prediction tool for cleavage by human and yeast 20S proteasomes, based on experimental cleavage data (http://www.paproc2.de/paprocl/paprocl.html) and (3) TEPITOPE program for prediction of MHC-II binding peptides. This program was available from Dr. Jurgen Hammer (Roche). (www.vaccinome.com) (17,18).

To identify the predicted proteasome-generated and MHC-I binding peptides, we downloaded the amino acid sequences of Notch1, Notch2 and Numb 1 from NCBI. Their accession numbers are: Notch1 (NM_{—}017617), Notch2 (NM_{—}024408), and Numb1 (P49757), respectively. We identified the peptides produced by the human proteasomes wild-type 1, 2, and 3.

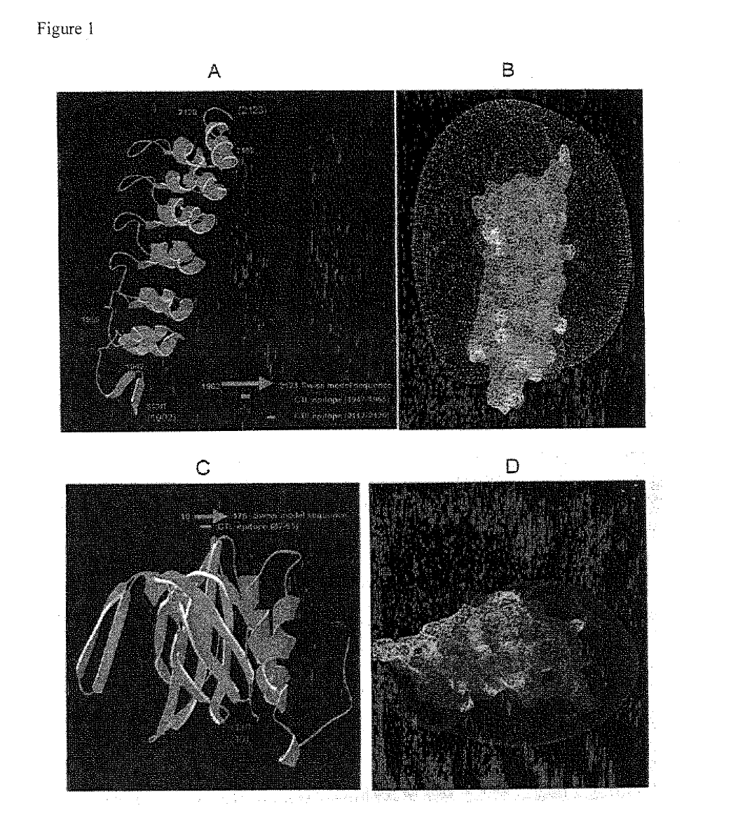

The tridimensional protein structure models of the Notch1 and Numb1 areas containing the peptide candidate CD8⁺ cells epitopes were down-loaded using the Swiss Model Program. The Swiss Model Program is a fully automated protein structure homology-modeling program, accessible via the ExPASy web server (http://swissmodel.expasy.org/repository/) or from the program Deep View (Swiss Pdb-Viewer, http://swissmodel.expasy.org/spdbvl) (19). The molecular models of the Notch1 and Numb1 regions where the peptides are located are shown in FIG. 1 (A-D) (20-22).

Cell Lines. We used the human breast cancer cell line MCF7, human ovarian cancer cell line SK-OV-3, and human leiomyosarcoma cell line SK-LMS-1 obtained from the American Type Culture Collection (Rockville, Md.). All cell lines were grown in RPMI 1640 medium (GIBCO, Grand Island, N.Y.) supplemented with 10% FCS, 100 units/ml penicillin, and 100 μg/ml streptomycin. Cells were grown in monolayers to a confluency of 80% before treatment.

Lymphocyte culture. Lymphocytes were isolated by Ficoll-gradient centrifugation from heparinized ascites from HLA-A2⁺ ovarian cancer patients. After separation, we cultured lymphocytes with RPMI 1640 medium with 10% FCS and 300 IU of IL-2 (Biosource Camarillo, Calif.) for one week, as we described (23,24).

Synthetic peptides. The following peptides were used in this study: Notch1 (1947-1955, RLLEASADA), Notch1 (2112-2120, RLLDEYNLV), Numb1 (87-95, VLWVSADGL), Glil (580-588, GLMPAQHYL) and AESI (128-137, LPL TPLPVGL). All these peptides were synthesized by Dr. Martin Campbell at the Synthetic Antigen Core Facility, of the University of Texas M.D. Anderson Cancer Center. Amino acids were coupled in sequential format from the COOH terminus using standard N-(9-fluorenyl) methoxy-carbonyl peptide chemistry on a Rainin Symphony Automated Peptide Synthesizer and purified by high-performance liquid chromatography. The purity of the peptides ranged from 95% to 97%. Peptides were dissolved in PBS with 10% DMSO and stored at −20° C. as aliquots of 1 mg/ml until use as we described (23).

Flow cytometry. To examine the expression of Notch1 molecules on tumor cell lines, cells that were pre-treated by BD Cytofix/Cytoperm and washed by BD Perm/Wash (BD Bioscience Pharmingen, San Diego, Calif.) for intracellular staining were stained with anti-Notch1 monoclonal antibody-PE (phycoerythrin)-labelled and PE-conjugated mouse monoclonal isotype control antibody (BD Bioscience Pharmingen) were analyzed using a Becton Dickinson FACS Caliber with Cell Quest software (Becton Dickinson, NJ) and the Flow-Jo Program (Mac version 8.11 Tree Star, Inc, OR) (25).

We identified cells expressing high concentrations/numbers of T cell receptors (TCRs) reactive with each peptide to evaluate the role of TCR density in CTL differentiation upon in vivo stimulation with the same ligands. The TCR⁺ population which usually includes cells staining with antigen-tetramers/dimers with a mean fluorescence intensity (MFI) higher than 101, was divided in three populations, one staining with antigen-pulsed HLA-A2/IgG dimers (dimers) with a MFI (TCR) between 101 and 102, and other which stained with antigen-pulsed dimers with a MFI (TCR) between 102 and 103, and other which stained with antigen-pulsed dimers with a MFI (TCR) between 103 and 104. These populations were designated as TCR^{lo}, TCRmed, and TCR^{med}, respectively, as we described (26).

T cell: peptide-HLA-A2-lgG dimer interaction. Expression of TCRs specific for peptides Notch1 (1940-1948), Notch1 (2112-2120), Numb1 (87-95), Gli1 (580-588) and AESI (128-137) was determined using HLA-A2-IgG-dimmers (BD Bioscience Pharmingen). The peptide loaded dimers were prepared as we previously described (23). Staining of lymphocyte with dimers was performed as described previously (24,27,28).

The same cells were also stained for the expression of CD8 antigen and the presence of Perforin, (effector pore forming enzyme) using specific antibodies conjugated to distinct fluorochromes than the dimers: fluorescein isothiocianate (FITC), allophycocyanin (APC) and PE. Cells reacting with the corresponding peptide-loaded dimers are designated as Notch1-1940-TCR⁺, Notch1-2112-TCR⁺, Numb1-87-TCR⁺, and Glil-87-TCR⁺ cells, respectively. Cells reacted with control HLA-A2-IgG dimers not loaded with peptide are designated as dNP-TCR⁺ cells.

### Results:

Selection of proteasome processed peptides. A preliminary analysis of the candidate immunogenic Numb and Notch peptides identified the peptides from Notch1, Notch2, and Numb1 which, based on the HLA-A, B, C binding-prediction algorithm, would bind to HLA-A, B, C molecules. Results show a very large number of peptides, which are potential binders to several MHC-I. The very large number of MHC-I binding peptides made peptide selection difficult. We searched and identified the peptides with potential to bind to: (a) HLA-A2, which is more frequently expressed in Caucasians and Chinese, (b) HLA-A24, which is more frequently expressed in Japanese, and (c) the HLA-A33, and HLA-Cw4, which were reported to be associated with T cell responses to HIV in African Americans (29). We also investigated the potential binders to HLA-A2.5 which is more frequent (25%) in HLA-A2⁺ African-Americans than in other HLA-A2 populations (30).

The immunodominance of self-/tumor (TA)-antigens, it is not always determined by the binding affinity of the antigen to MHC-I. In fact, some of the immunogenic peptides (C85, MART-I) are very weak binders to HLA-A2. To improve our chances of selection of immunogenic peptides, which are endogenously processed, we performed proteasome-digestion prediction analysis (18). Results in Table I show that only very few Notch1, Notch2, and Numb 1 peptides of the ones predicted to bind any of the HLA-molecules can be also generated by proteasomal digestion of internal proteins. In fact, only two peptides from Notch 1, and one from Numb 1 were similar with their MHC-I-predicted to bind, counterparts.

TABLE I
Proteasome generated Notch1, Notch2 and Numb1 peptides^{a}

| | | | | | |
|---|---|---|---|---|---|
| | Start | | Digestion | | |
| HLA- | position | Sequence | type^{b} | Digestion product^{c} | Length |

Notch 1

| | | | | | |
|---|---|---|---|---|---|
| A2.1 | 1947 | RLLEASADA | 1 | AAKR/LLEASAD/A | 7 |
| A2.1, 2.5 | 2112 | RLLDEYNLV | 1 | VR/LLDEYNLV | 8 |
| A24 | 1938 | RYSRSDAAK | 1 | RYSRSD/AAKR | 6 |
| A33 | 274 | DGVNTYNCR | 3 | DGVNTYNC/R | 8 |
| Cw4 | none | N/A^{d} | | N/A | |

Notch 2

| | | | | | |
|---|---|---|---|---|---|
| A2.1 | none | N/A | | N/A | N/A |
| A2.5 | 7 | ALLWALLAL | 1, 2 | MPALRPALLWALLALWLCCA | 21 |
| A24, 2.5 | 1940 | RMNDGTTPL | 3 | RMNDGTTPLI | 10 |
| A33 | 1995 | LLLKNGANR | 1 | EATLLL/LKNGANR | 7 |
| A33 | 277 | DGVNTYNCR | 2 | DGVNTYNCRCPPQWTG | 16 |
| | 277 | DGVNTYNCR | 3 | NGGVCVDGVNTYNC/R | 14 |
| Cw4 | none | N/A | | N/A | |

Numb1

| | | | | | |
|---|---|---|---|---|---|
| A2.1 | 87 | VLWVSADGL | 1 | V/LWVSADGL | 8 |
| A2.1, 2.5 | 443 | WLEEVSKSV | 2 | RWLEEVSKSVRA | 12 |
| A2.5 | 139 | WICHCFMAV | 1 | RWICHCFMAVKD | 12 |
| | 139 | WICHCFMAV | 2 | CRDGTTRRWICHCFMAVKD | 19 |
| A24 | none | N/A | | N/A | N/A |
| A33 | 594 | DGRLASADR | 1 | VDDGRLASADRHTEV | 15 |
| Cw4 | none | N/A | | N/A | N/A |

^{a)}The predicted proteasome generated peptides which can bind MHC-1 were identified with the program PAPROC (http://www.paproc2.de/paproc1/paproc1.html)
^{b)}Digestion type indicate the proteolytic sperificities, designated as 1, 2, and 3 by the program PAPROC
^{c)}“/” represents the positions of digestion of peptide and the resulting product.
^{d)}N/A indicates, “not applicable” no peptides binding to

Results in Table I show that peptides Notch1 (2112-2120) and Notch1 (274-282) are processed by the proteasome and presented as octamers, by HLA-A2 and HLA-A33, respectively. Based on the position of N and C-terminal anchor motifs, only Notch1 (2112-2120) can form a complex with HLA-A2. Of interest, Notch1 (2112-2120) can also bind A2.5, although with lower affinity, than HLA-A2.1. Therefore, Notch1 (2112-2120) can be a common/shared epitope for Caucasian and African-American populations, which express A2.1 and A2.5 respectively.

Completely different results were obtained for Notch2 peptides. Only the peptide Notch2 (19401948) can be digested by the proteasome and presented as a decamer by HLA-A24. This peptide and all other Notch2 peptides cannot be presented by HLA-A2 or any of the histocompatibility gene products associated with responses in African-American populations. However, Notch2 (1940-1948), can be generated by proteasome and presented by HLA-A2.5. Therefore, the Notch2 (1940-1948) can be presented by tumors in association with both HLA-A24 and HLAA2.5. It should be also emphasized that Notch2 (1940-1948) differs in sequence from Notch1 (1947-1955).

Results were surprising for Numb. The Numb1 peptide (87-95) can be digested by the proteasome and presented as an octamer by HLA-A2.1. The Numb peptide 443-451 can be presented by HLA-A2.1 and HLA-A2.5 as a dodecamer, thus its immunogenicity may depend on trimming by exopeptidase.

Detection of naturally immunogenic peptides. To address whether the peptides imperfectly digested by the proteasome can be repaired, we engineered new candidate immunogens. Peptides which exceed the 9-amino acids length such as Notch2 (1940-1948) and Numb (443-451) can be trimmed at N- and C-terminal ends before presentation. To engineer repairs, we kept the same minimal nine amino acid epitope and modified the flanking residues. Modification was made by replacing the Notch/Numb flanking residues with the flanking residues from other proteins (e.g. HER-2 protein) which allows presentation of the minimal CTL epitope, E75, associated with HLA-A2. Results show that only the HLA-A2 binding peptides from Notch1 and Numb1 could be presented after proteasome digestion (Table II).

TABLE II

Repair of proteasome generated peptides by modification of flanking residues
of the core peptide

| | | | | |
|---|---|---|---|---|
| Peptide | Flank | Core | Flank | Proteasome Digestion Product |

Notch 1

| | | | | |
|---|---|---|---|---|
| Wild-type | RMHHDI | VRLLDEYNLV | RSPQL | RMHHD/I/VR/LLDEYNLV/RSPQL |

A. Replace N-terminal flanking sequence with the Her-2 E75
peptide N-terminal flanking sequence NIQEAFAGCL

| | | | | |
|---|---|---|---|---|
| N-flank-modified | NIQEAFAGC | LRLLDEYNLV | RSPQL | NIQEAFAGC\|L\|RLLDEYNLV\|RSPQL |

B. Replace N-terminal flanking sequence with NIQEAFAGCL
and then replace in the core: R² with K

| | | | |
|---|---|---|---|
| NIQEAFAGC | L [symbol] LLDEYNLV | RSPQL | NIQEAFAGC\|L\| [symbol] LLDEYNLV\|RSPQL |

Numb1

| | | | | |
|---|---|---|---|---|
| Wild-type | GKTGKKAVKA | VLWVSADGL | RVVDEKTK | GKTGKKA\|V\|KA\|V\|LWVSADGL\|RVVDEKTK |

Substitutions (**)

| | |
|---|---|
| A → P | GKTGKKA\|V\|K\| [symbol] VLWVSADGL\|RVVDEKTK |
| KA → LFK | GKTGKKA \|V\| [symbol] \| [symbol] VLWVSADGL\|RVVDEKTK |

Replace the N and C-terminal flanking residues wih RMHHDI and
RSPQL respectively * plus insert R before the start of the minimal epitope

| | | | |
|---|---|---|---|
| RMHHDIA VR | VLWVSADGL | RSPQL | RMHHDI\|AV\|R\|VLWVSADGL\|RSPQL |

(*) RMHHDI and RSPQL are the flanking residues of the Notch1 peptide above.
(**) All resulting peptides have very low affinity for HLA-A2.
HLA-A2 binding scores are: 147.697 (9mer), 0.075 (10mer) and 11.861 (10mer).
Bold and italicized letters indicate substitutions in the sequence.

To identify which of these proteins is antigenic in vivo, we determined the presence of CD8⁺ T cells expressing TCRs which can specifically recognize peptides Notch1 (1947-1955), Notch1 (2112-2120), and Numb1 (87-95). The AESI peptide (128-137), which is known to be generated by proteasomal digestion, was used as negative control for in vivo immunogenicity. The Gli 1 peptide (580-588), which is not generated by proteasomal digestion, was used as a negative control. The base line TCR⁺ cell numbers were determined with dNP-dimers. We investigated the presence of CD8⁺ cells bearing TCRs with high, medium and low affinity in ovarian tumorassociated lymphocytes from patients with advanced disease.

The significance of the presence of Notch and Numb proteins and ligands in ovarian cancer, due to the fact that Notch and Numb are expressed in a subset of ovarian vessels during oncogenesis, including both mature ovarian vasculature as well as angiogenic neovessels (31). Their expression in the ovary was found in both endothelial and vascular associated mural cells (32) Tumor angiogenesis involves many of the same pathways as physiological angiogenesis, including Notch. This has been shown in both human tumor samples and mouse xenografts. Measured by in situ hybridization and puantitative polymerase chain reaction (qPCR), 0114 mRNA was undetectable in normal kidney or breast samples, but highly expressed in the vasculature of human clear-cell renal cell carcinomas and breast cancers. Among the tumor samples, 0114 expression positively correlated with YEGF expression at the mRNA level (33). In a xenograft study, the human MCF7 cell line, which does not express 0114, resulted in tumors. expressing high levels of mouse 0114 within their vasculature (34). Currently, the study of 0114 expression in tumors is hampered by the lack of a good monoclonal antibody. Work is underway to develop antibodies that allow measurement of 0114 protein levels by immunohistochemistry.

Elements of the Notch pathway regulate differentiation are expressed more frequently in adenocarcinomas whereas Deltex, Mastermind were more frequent in adenomas (35). qPCR revealed decreased Notch1 mRNA in ovarian adenocarcinomas compared with adenomas. The expression of Notch1-extracellular protein was similar in benign and malignant tumors (35). HES-1 protein was found strongly expressed in 18/19 ovarian cancers and borderline tumors but not in adenomas. Thus, some of the Notch pathway elements are differentially expressed between adenomas and carcinomas (36).

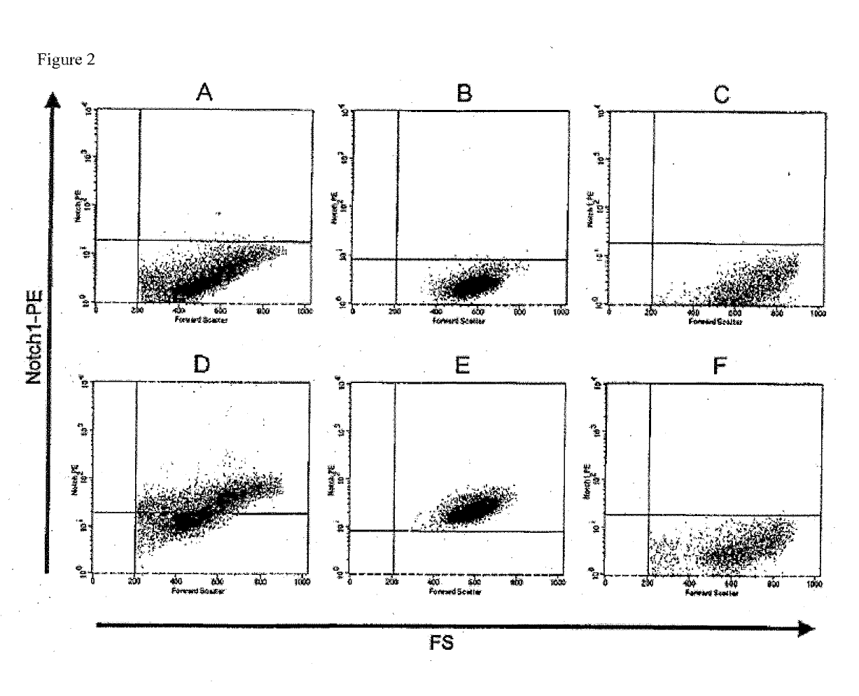

In separate experiments, we found that AES1 is strongly expressed in SK-OV-3 (ovarian cancer cells) and SKBR3 (breast cancer cells). To examine the expression of Notch1 on tumor cell, we stained SK-OV-3, MCF7, and SK-LMS-1 malignant leiomyosarcoma cells with antibodies against Notch1 and corresponding isotype controls. Results in FIG. 2 (A-F) show that SK-OV-3 and MCF7 express Notch1, but SK-LMS-1 does not express Notch1.

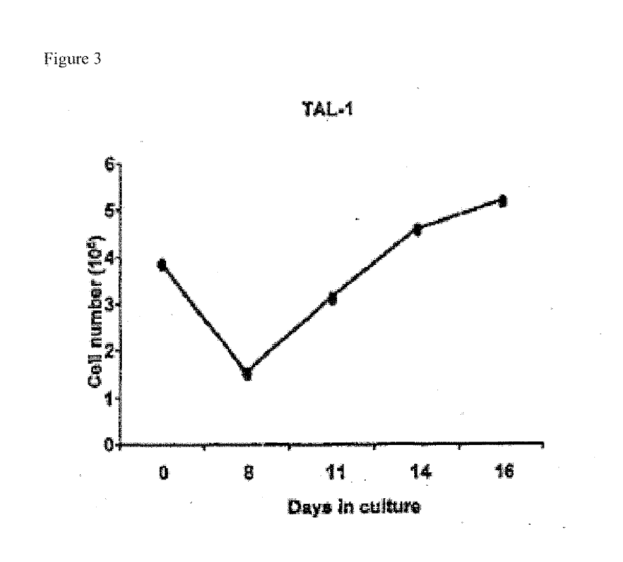

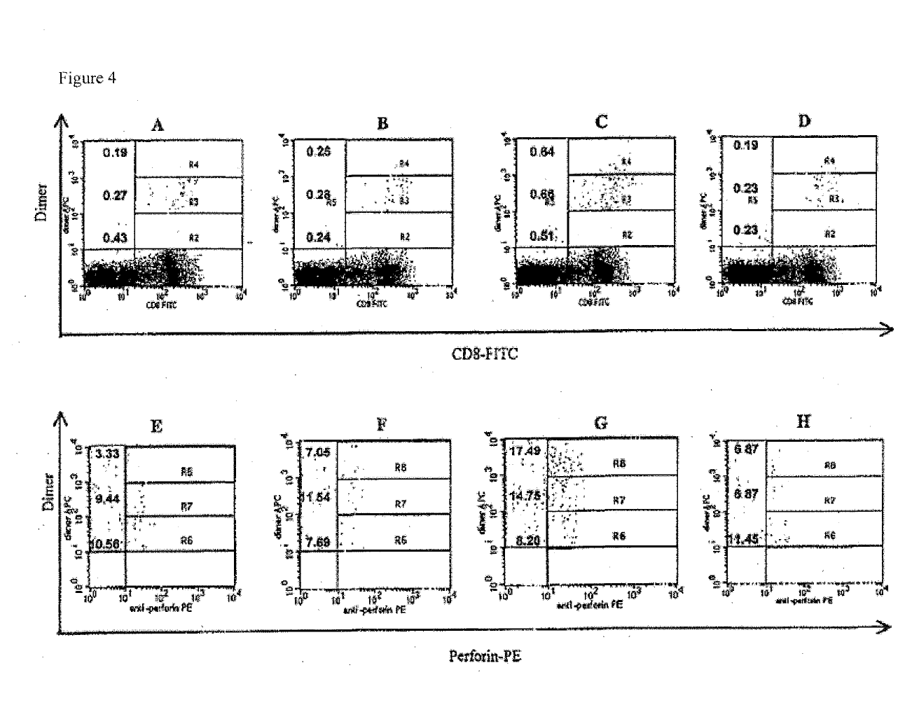

We cultured ovarian ascites with low concentrations of IL-2 to avoid expansion of non-activated clones. FIG. 3 shows the kinetics of growth of tumor associated lymphocyte (TAL). We found that CD8⁺ Numb1-87-TCR⁺ cells were present in cultured ascites from patient No. 1, in higher numbers than the Notch1-2112-TCR⁺, and AES1-128-TCR⁺ cells (FIG. 4B-D). Numb-TCR⁺ CD8⁺ cells expressed Perforin indicating that these cells were differentiated in vivo (FIG. 4G). It should be mentioned that expression of Perforin is controlled by two main signals: one from TCR and the other from IL-2. Since T cells of all specificities were cultured in the same amount of IL-2, our results indicate that differences in Perforin expression were due to activation by antigen.

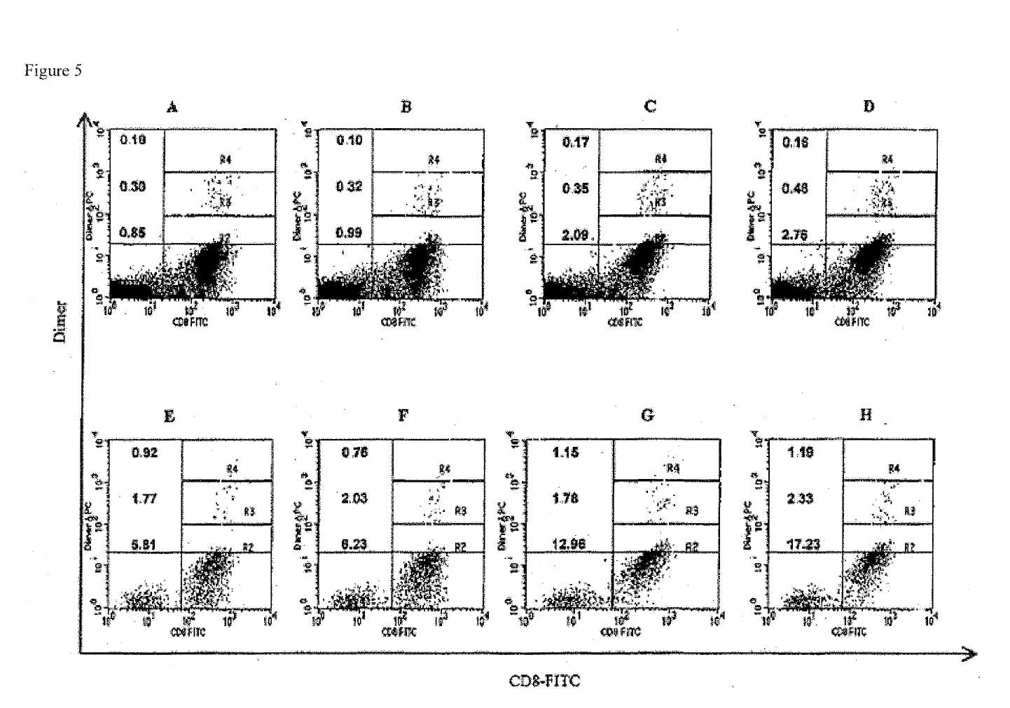

To address whether Notch1-TCR⁺ and Numb-TCR⁺ cells are present in ascites from other patients, we repeated the experiment with ovarian-TAL from four additional HLA-A2⁺ patients. Table III, and FIG. 5 show that ascites from Patients No 2, 4, and 5 contained Notch1-2112TCR⁺, and Numb1-87-TCR⁺ CD8⁺, cells. Notch1-2112-TCR⁺, Numb1-87 TCR⁺ cells were no longer detected in the cultured ascites from Patient 3 after two weeks culture with IL-2, (Table III), indicating that these cells either did not expand or they were diluted because of outgrowth of other T cell populations.

TABLE III
The Notch1 and Numb1-TCR⁺CD8⁺ populations
based on the density of the specific TCR

| | | % TCR⁺ cells for HLA-A2: peptide | | | | |
|---|---|---|---|---|---|---|
| Patient | TCR-density | NP | Notch1-1947 | Notch1-2112 | Numb1-87 | AES1 |
| 1. | High | 0.19 | N.D. | 0.26 | 0.64* | 0.19 |
| | Med | 0.27 | N.D. | 0.28 | 0.66* | 0.23 |
| | Low | 0.43 | N.D. | 0.24 | 0.51 | 0.23 |
| 2. | High | 0.10 | 0.10 | 0.17 | 0.16 | N.D. |
| | Med | 0.30 | 0.32 | 0.35 | 0.46 | N.D. |
| | Low | 0.85 | 0.99 | 2.09* | 2.76* | N.D. |
| 3. | High | 0.09 | 0.10 | 0.08 | 0.09 | N.D. |
| | Med | 0.22 | 0.24 | 0.28 | 0.21 | N.D. |
| | Low | 0.51 | 0.65 | 0.43 | 0.50 | N.D. |
| 4. | High | 0.11 | 0.22 | 0.08 | 0.22 | N.D. |
| | Med | 0.13 | 0.26 | 0.34* | 0.26 | N.D. |
| | Low | 0.84 | 0.53 | 0.88 | 0.53 | N.D. |
| 5. | High | 0.11 | 0.14 | 0.17 | 0.27* | N.D. |
| | Med | 0.22 | 0.26 | 0.36 | 0.27 | N.D. |
| | Low | 1.98 | 1.98 | 2.52 | 1.84 | N.D. |

*significantly higher (2-fold) than the % positive cells reactive with base line control dNP and higher than the specificity control Notch1(1947)-TCR⁺ cells. Ovarian TALs were cultured for one week in medium containing with 300 IU IL-2.

To characterize the CD8⁺ populations based on the density of the specific TCR, we investigated the presence of TCR^{hi}, TCR^{med}, and TCR^{lo} cells. FIGS. 5D and H show the presence of a significant number of Numb1-87-TCR^{lo} CD8⁺ cells in Patient-2, compared with controls, cells interacted with base-line control, empty dimers (dNP-TCR⁺ cells) and cells interacted with HLA-A2 dimers pulsed with negative control, Notch1-1947 peptide. There was also a small increase in Notch1-2112-TCR⁺ cells (FIGS. 5C and G). These results were confirmed at a separate analysis of CD8⁺ cells, in the large-blast-size population (FIGS. 5G and 5H). The large blastsize T cells are lymphocytes with active cellular synthesis and divide. Similar results were observed with Patient 5, with the difference that in this patient Numb1-87-TCR^{hi} CD8⁺ cells were 2.45-times more than cells reactive with control, dNP-HLA-A2-IgG dimers. Notch 1-2112TCR^{med} cells were also present in 1.63 times higher number than cells reactive with the base-line control, dNP (Table III). In the Patient 4, we found 2.61-times more Notch1-2112-TCR^{med} cells compared with cells interacted with the base-line, NP dimers (Table III). These results show that all ascites from all four ovarian patients contained cells bearing TCR for Notch1-2112 and/or for Numb 1-87 peptides.

Therefore peptides Notch 1-2112 and Numb1-87 not only are generated in vivo, but also activate CD8⁺ cells in vivo in the ascites of ovarian cancer patients.

Discussion: In this study, we identified candidate peptides from Notch and Numb, which are natural immunogens in vivo for CD8⁺ cells in ovarian cancer patients. The candidate peptides were selected based on their binding motifs to the HLA-A2, HLA-A24, HLA-A33, and HLA-Cw4 molecules. As an additional parameter of stringency, we identified the candidate naturally immunogenic peptides produced by the proteasome. Third, of the peptides identified to be produced by the proteasome, we selected only the “reparable” peptides. Only “reparable” peptides can be expressed by DNA and RNA vectors which deliver the precursor of tumor Ag in APC.

Surprisingly, we found very few naturally immunogenic peptides from each protein and only one each to be presented in association with HLA-A2. The naturally immunogenic peptides were identified by a novel and sensitive method. We used TA/peptide loaded HLA-A2-lgG dimers, and we determined the specificity of recognition of the ovarian TAL by comparing the staining with negative control dimers which were not loaded with peptides. Differentiation of these lymphocytes was determined by measuring expression of Perforin and the amount of Perforin (as MFI) per cell. We found that two of five patients had activated CD8⁺ Perforin⁺ cells expressing TCR specific for the Notch1-2112 peptide and three of five have activated CD8⁺ Perforin⁺ cells expressing TCR specific for the Notch1-87 peptide. These CD8⁺ cells expressed a higher density of TCRs than the known low TCR density of T cells recognizing tumors. Our results predict the use of Notch1-2112 peptide and Numb 1-87 peptide for ovarian cancer immunotherapy.

Notch and Numb are expressed not only in ovarian cancer cells but also in breast, pancreas, liver, stomach and colon cancers (5-7,37). Specific immunotherapy targeting these molecules can be effective in elimination of tumors which express those antigens. Recently, Notch and Numb were shown to control differentiation and the metastatic potential of cancer cells. It is possible that that immunotherapy targeting Notch and Numb will became soon a therapeutic choice for cancers of the liver and pancreas which are not only chemotherapy resistant, but rapidly result in the death of patients.

Results of this study also indicate a selectivity of immunogenic TA towards the HLA-A2 system. The HLA-A2 supertype includes in addition to HLA-A2 (subtypes 1-7), HLA-A68.2, and HLAA69.1. However, when the results of proteasome digestion were compared with the affinity for HLA-A2 subtypes, only HLA-A2.5 could present the same peptide with HLA-A2.1. HLA-A2.5 is considered an ancestral allele, associated with human origins. However Numb1 peptides which can be presented by HLA-A2.5 do not appear to confer protection to cancer. Only Notch2 peptides associated with HLA-A2.5 and HLA-A24 may confer some protection. Is then Notch2 significant for cancer prevention in some of African-Americans, while Notch1 significant for prevention in Caucasians?

The association of Notch1 and Numb1 with HLA-A2.1 may be significant for cancer prevention in Caucasians and Hispanics. Is then protection from liver and pancreatic cancer due to the “redundancy” of the immunosurveillane first by Numb 1 and then by Notch 1?

Peptides binding to HLA-A24 were negatively selected for presentation. We found only the decamer Notch2 (1940-1949), as both potentially binding to HLA-A24 and produced by proteasome digestion. None of the Notch1 and Numb1 peptides associated with HLA-A24 was positively selected. The HLA-A24 product is frequently preset in South-East Asian, especially it is most frequent in Japan (38).

There are clear differences in cancer incidences among different ethnic groups. For example, there is at least a 25-fold variation in occurrence of colorectal cancer worldwide. The highest incidence rates are in North America, Australia/New Zealand, Western Europe, and, in men especially, Japan (49.3 per 100,000); incidence tends to be low in Africa and Asia (e.g., China 13.6 per 100,000 in men) and intermediate in southern parts of South America. For gastric cancer, geographical distribution of stomach cancer is characterized by wide international variations; high-risk areas include East Asia (e.g., Japan—age standardized rate 62.1), Eastern Europe, and parts of Central and South America. Incidence rates are low in men in Southern Asia, North and East Africa, North America (e.g., age standardized rate of only 7.4), and Australia and New Zealand. The incidence of pancreatic cancer is highest among USA and Japan (11.8 and 10.9 per 100,000 respectively), while it is lowest in Africa and China (2.1 and 6.3 per 100,000, respectively). Many factors could have contributed to the wide variation, e.g., diet, environment, habits (smoking and drinking history), and genetics. Immunegenetics could certainly be one of the contributing factors (39).

Such factors may include the composition of the diet, and at the same nominal composition of the diet, the presence in the diet of compounds which interfere with metabolic or tissue regeneration pathways.

Development of immunotherapy against Notch1 and Numb with peptide vaccines may be useful for populations at high risk of developing rapidly deadly cancers.

Park et al recently reported that Notch-3 is overexpressed in ovarian cancer (37). We found 6 Notch-3 peptides that bind to HLA-A2 molecules and are digested by proteasome type I enzymatic activity, but few or none digested by protesome type II, or type III. Notch-3 peptides may be good targets for cancer immunotherapy.

### Example 2

### Introduction

During normal development stem-cell renewal is regulated by signals from the surrounding stem cell environment. Expansion of the stem-cell population stops when a specific niche or an organ is formed. This event does not imply metastatic transformation, since a large number of benign tumors can expand for similar reasons. Elucidation of the mutual impact of pathways that regulate the self-renewal of normal cells, such as Notch and Hedgehog is ongoing (40).

Cancer cells contain deregulated Notch and Hedgehog pathways together with activated oncogenes (such as Ras, BCr-Abl, etc). Although chemotherapy and radiotherapy are expected to eliminate tumor cells, metastases suggests that tumor cells having characteristics of cancer stem cell (CSt-C) are hiding in the population of chemotherapy- and radiotherapy-resistant tumor cells. The proliferating potential of cancer cell is very similar to the ability of normal stem cell. This potential could be explained as symmetric cell division, and anchor-independent cell growth (41). It is likely that normal stem cell change into malignant stem cell (Cancer stem cell) when accumulate oncogenic Ras-mutations (42).

Pancreatic cancer (PC) is the fifth most common cancer worldwide. The reasons for its very high mortality rate include the lack of early diagnosis, the unresectability at the time of initial diagnosis, and the rapid recurrence after resection. Surgical resection is rarely a curative option in pancreatic cancers because of local extension and metastases. For patients with advanced pancreatic cancer, the treatment options such as chemotherapy are limited, with gemcitabine (GEM) the current standard therapy (43, 44). Many clinical trials investigated combination chemotherapies, but none has identified a strategy that offers a significant improvement for the prognosis of advanced pancreatic cancer patients. New therapeutic approaches are needed (45-49). One break-through point may be targeting CSt-C resistant to chemotherapy.

Breast cancer cells (BR-C) characterized by the expression of cell surface markers CD44 and CD24^{dim} (CD24^{low}) have CSt-C functional characteristics (50). CD44⁺ CD24⁺ ESA⁺ pancreatic cancer cells formed tumors in immunocompromised mice (51). CD44 might be important for CSt-C because the levels of CD44 correlated with homing of cancer cells during metastasis (52). Expression of CD133 (Prominin-1) distinguished between neural St-C and brain CSt-C (53). CD133⁺ colon cancer cells grew exponentially unlike CD133⁻ cells (54, 55). Normal prostate stem cells also express CD133, however prostate cancer cells with CD44⁺/α2β1^{high}/CD133⁺ phenotype have CSt-C characteristics (56).

These findings raised the question whether chemotherapeutic agents eliminate cells expressing CSt-C markers. We found that GEM positively selected CD44⁺ CD133⁺, and CD24^{low} CD133⁺ cells in PC, BR-C, and epithelial ovarian cancer (EOVC) lines. GEM-resistant (GEM^{Res}) PC, MIA-PaCa-2 differed in expression of NECD and NICD from GEM^{Res} BR-C, MCF7. DLL4-activation of GEM^{Res} cells resulted in 2-3 fold higher expansion of CD44⁺ CD24^{low} cells than medium containing. Notch⁺ and CD44⁺ CD24^{low} cells were eliminated by Notch and Numb peptide-activated PBMC and at lesser extent by IL-2 activated PBMC.

Materials and Methods

Cell lines and materials. The human cancer lines PC (MIA-PaCa-2, PANC-1, and AsPC-1), BR-C cell line (MCF7), ovarian cancer (SKOV-3) were purchased from American Type Culture Collection (ATCC; Manassas, Va.). All cells were cultured in the RPMI 1640 medium supplemented with 10% fetal calf serum (FCS), 100 U/L penicillin and 100 μg/mL streptomycin, in a 95% humidified air and 5% carbon dioxide at 37° C.

Reagents were purchased as follows: gemcitabine hydrochloride (Gemzar®, Eli Lilly and Co., Indianapolis, Ind.), paclitaxel (Taxol®, Bristol-Myers Squibb Co., Princeton, N.J.), 5-fluorouracil (5-FU, Sigma, Saint Louis, Mo.), Fluorescein isothiocyanate (FITC)-conjugated mouse anti-human epithelial specific antigen (ESA) monoclonal antibody (Biomeda, Foster City, Calif.), Allophycocyanin (APC)-conjugated mouse anti-CD44 monoclonal antibody (BD Pharmingen, San Diego, Calif.), FITC-conjugated mouse anti-CD44 monoclonal antibody (BD Pharmingen, San Diego, Calif.), R-Phycoerythrin (R-PE)-conjugated mouse anti-CD24 monoclonal antibody (BD Pharmingen, San Diego, Calif.), FITC-conjugated mouse anti-CD24 monoclonal antibody (Abcam Inc., Cambridge, Mass.), PE-conjugated mouse anti-MICA/B antibody (R&D Systems, Inc., Minneapolis, Minn.), APC-conjugated mouse anti-CD133/2 antibody (Miltenyi Biotec Inc., Auburn, Calif.) and recombinant human Delta-like protein 4, (DLL4) (R&D Systems, Inc., Minneapolis, Minn.).

Inhibition of proliferation of tumor cell lines by anticancer drugs. The IC50 was determined by the classical 3-(4,5-dimethylthriazolyl)-2,5-diphenyl-tetrazolium bromide (MTT) assay after 72 hours exposure with GEM, PTX and FU as we described (73).

Flow cytometery analysis. All cells were cultured with Gem at 2×IC50 of gemcitabine for 10 days. Cultured cells (2×10⁵) were washed in cold-PBS followed by blocking with 20 μL of 1 mg/mL of human IgG (Sigma, Saint Louis, Mo.) for 1 hour on ice. This step was necessary to inhibit non-specific binding of immunoglobulins during staining. Cells were then triple-stained with antibodies against ESA, CD44, and CD24. Analysis was performed with Becton Dickinson FACSCalibur and Cell Quest software (Becton Dickinson). Cells were gated on ESA+ population. Expression of CD24 and CD44 was examined in gated ESA+ cells as we described (26). The population of the ESA+, CD44hi and CD24low/− cells was calculated as percent of total cells and total ESA+ cells. All cell lines were also stained with a MIC-A/B and CD133, and analyzed as above. In other experiments MIA-PaCa-2 and MCF7 were cultured with 2-fold IC50 concentration of GEM, PTX, or FU for 4 days followed by 0.7-fold IC50 concentration for 3 days, and stained and analyzed as above.

Stimulation of GEMRes MCF7 by DLL4. GEMRes MCF7 were obtained after culture with 0.3 uM GEM for 7 weeks. MCF7 were stimulated for 24 hrs, in medium containing estradiol, fibroblast growth factor in the presence or absence of DLL4, as described (40).

Stimulation of HLA-A2 PBMC with Notch and Numb peptides. Naturally immunogenic NotchNICD (2112-2120) and Numb 1-PTB domain peptide (87-95), were identified as we described (Ishyiama 2007). Non-adherent PBMC were activated with peptide-pulsed autologous immature DC as we described (26).

Western blot analysis. Cell lysates of live MIA-PaCa-2, MCF7, and SKOV-3 were prepared as we described (74) after trypsin treatment of cultures. This procedure eliminated dead and dying cells. Cellular proteins were resolved by SDS-PAGE and transferred to a polyvinylidene difluoride membrance. Immuno-blotting and quantification was performed as we described (74).

Results

The drug sensitivity of PC lines Mia-PaCa-2 and PANG-1 is similar to that of BR-C line MCF7. To select anticancer drug resistant cells, we quantified the cytotoxicity of GEM, 5-fluoruracil (5-FU), and paclitaxel (PTX) on the PC lines MIA-PaCa-2, PANC-1, AsPC-1; the BR-C line, MCF7; and the EOVC line, SKOV-3. All 3 drugs are effective for cancer treatment. GEM provides a little better clinical benefits against PC than 5-FU in Phase III trials (44, 45). PTX was also tried against PC but did not show improvement compared with GEM.

Table 1 shows the drug concentrations that inhibited cell proliferation by 50% (IC₅₀) in 72 h. The widest variance in the IC₅₀ was found for 5-FU ranging from 800 (PANC-1) to 15,200 nM (AsPC-1). IC₅₀ for PTX was in a narrow range from 3.9 to 18.3 nM. The IC₅₀ in the most PTX-resistant AsPC-1 was more than 4-fold that of the most PTX-sensitive PANC-1. Mia-PaCa-2, PANC-1, and MCF7 displayed similar high resistance to GEM with IC₅₀ of 300, 350, and 430 nM respectively. AsPC-1 and SKOV-3 were GEM-sensitive (GEM^{Sens}) with IC₅₀ under 20 nM. Therefore the IC₅₀ of three drugs in Mia-PaCa-2, PANC-1, and MCF7 was similar.

TABLE I
A. IC₅₀ of gemcitabine, 5-fluorouracil, and paclitaxel

| Cell lines | IC₅₀ (nM) GEM | 5-FU | PTX |
|---|---|---|---|
| MIA-PaCa-2 | 300 | 3,700 | 5.3 |
| PANC-1 | 350 | 800 | 3.9 |
| AsPC-1 | 20 | 15,200 | 18.3 |
| MCF7 | 430 | 1,300 | 4.5 |
| SKOV-3 | 16 | 3,600 | 4.7 |

B. Expression of Breast CSt-C markers after culture with chemotherapeutic drugs.

| | Treated | % | % CD44^{hi} CD24 in ESA⁺ cells | | | % CSt- |
|---|---|---|---|---|---|---|
| Cell line | with | ESA⁺ | CD24⁻ | CD24^{low} | CD24^{hi} | like-C |
| MIA-PaCa-2 | NT | 24.0 | 0.5 | 41.4 | 10.7 | 9.9 |
| | GEM | 39.5 | 1.1 | 43.2 | 12.2 | 17.0 |
| | PTX | 33.2 | 1.0 | 23.1 | 21.2 | 7.7 |
| | 5-FU | 83.0 | 0.2 | 19.8 | 7.1 | 16.4 |
| PANC-1 | NT | 50.8 | 49.9 | 35.5 | 11.3 | 18.1 |
| | GEM | 76.7 | 8.6 | 50.0 | 9.7 | 38.3 |
| AsPC-1 | NT | 98.4 | 25.3 | 56.4 | 17.8 | 55.5 |
| | GEM | 98.9 | 19.3 | 58.2 | 20.1 | 57.6 |
| MCF7 | NT | 98.2 | 0.0 | 1.3 | 15.6 | 1.3 |
| | GEM | 95.4 | 0.3 | 6.3 | 10.2 | 6.3 |
| SKOV-3 | NT | 99.7 | 0.0 | 4.6 | 95.1 | 4.6 |
| | GEM | 97.5 | 0.1 | 51.5 | 46.0 | 50.2 |

C. Notch ligand, DLL4, activate proliferation of MCF7 cells

| | Seeded | Harvested | Stimulation | CD44^{hi} | CD44^{hi} |
|---|---|---|---|---|---|
| Treatment | cells: 10⁶ | cells: 10⁶ | Index | CD24^{low} | CD24^{hi} ᵣₐₜᵢₒ |
| NT | 3.0 | 12.96 | 4.32 | 0.52 × 10⁶ | 3.89 |
| | | | | (4.0%) | (30.0%) = 7 |
| DLL4 | 3.0 | *18.80 | 6.27 | 0.66 × 10⁶ | 6.84 |
| | | | | (3.5%) | (36.4%) = 10.4 |
| GEM | 3.0 | 1.32 | 0.44 | 0.09 × 10⁶ | 0.26 |
| | | | | (7.1%) | (19.7%) = 2 |
| DLL4 GEM | 3.0 | *2.16 | 0.72 | 0.14 × 10⁶ | 0.41 |
| | | | | (6.7%) | (19.1%) = 2 |

*45%< increase in total cell number at stimulation with DLL4.
**2.7-2.8 fold increase in Population of BR-CSt-C after selection with gemcitibine compared to without gemcitabine.

TABLE 2
Antigen expression in cell lines

| | | Gli-1 | Gli-2 | |
|---|---|---|---|---|
| | HER-2* | (%) positive | (%) positive | |
| Cell lines | density(MFI) | cells | cells | HLA-A2 |
| MIA-PaCa-2 | 2+ (75.4) | 91.9 | 37.1 | + |
| PANC-1 | 1+ (29.3) | 43.9 | 15.9 | + |
| AsPC-1 | 1+ (33.4) | 74. | 5.7 | + |
| MCF7 | 3+ (1063.5) | 13.2 | 8.1 | + |
| SKOV-3 | 2+ (100.5) | 69.9 | 24.8 | + |

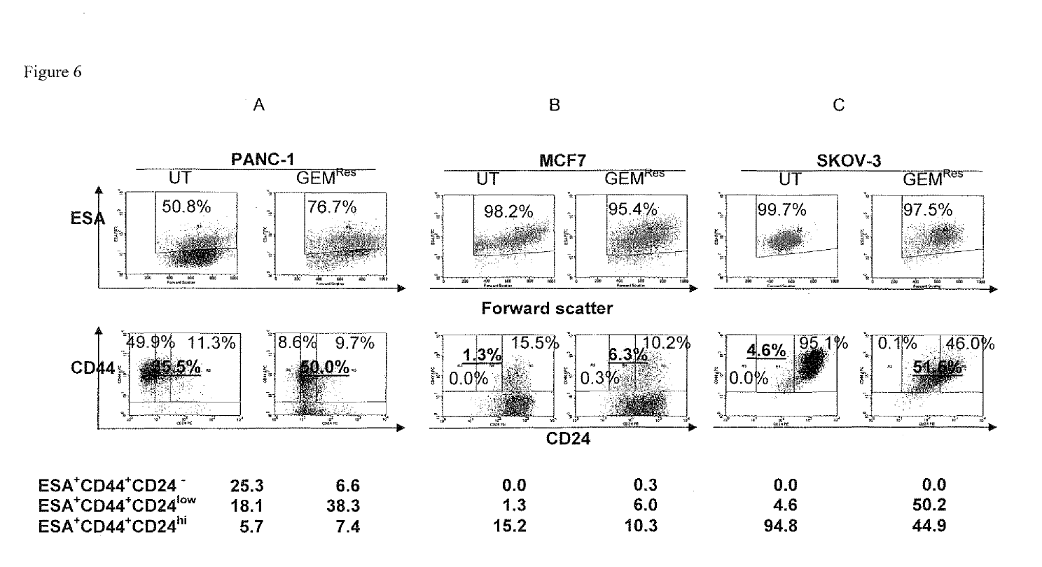

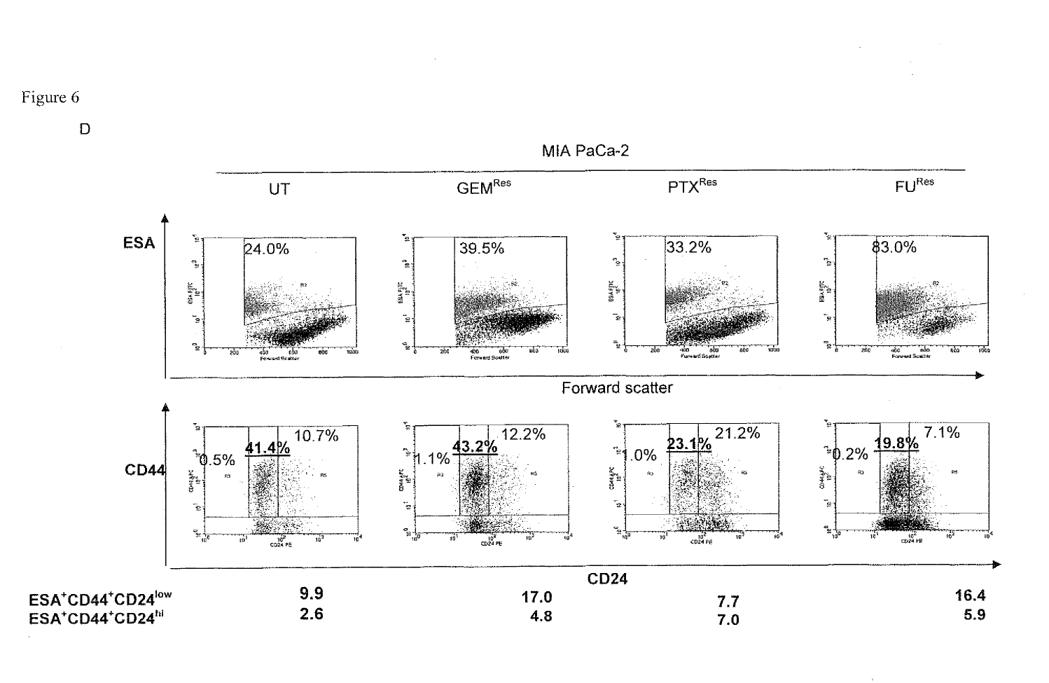

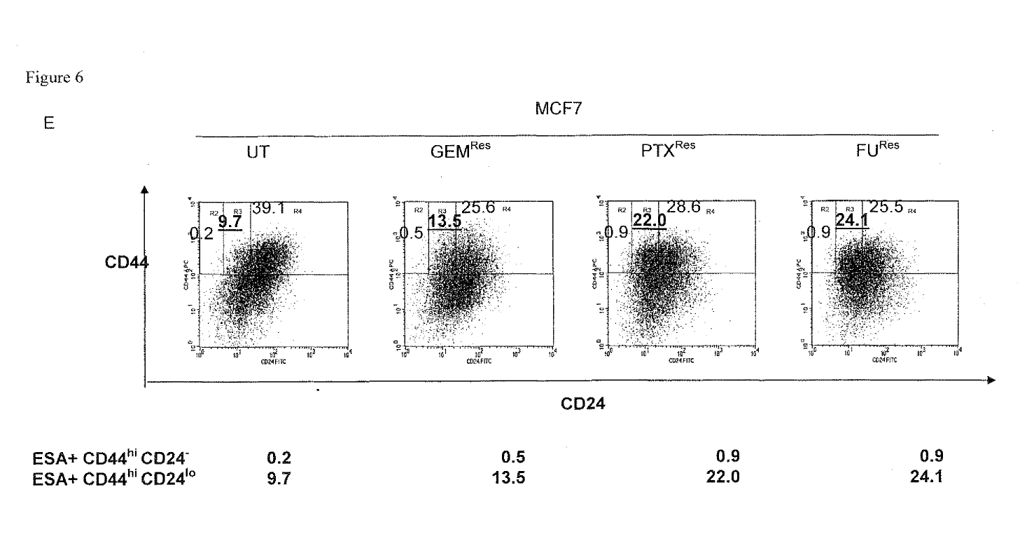

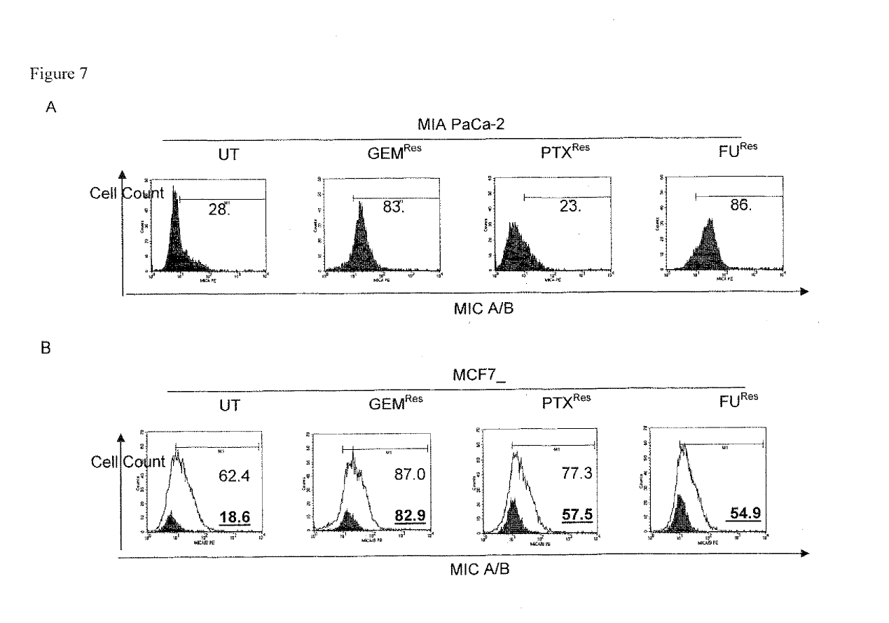

ESA⁺ CD44⁺ CD24^{low}, CD44⁺ CD133+ and CD24^{low} CD133⁺ cells increased in PC, BR-C, and EOVC resistant to drugs. ESA⁺ CD44^{hi} CD24^{low} cells from breast tumors have the functional characteristics of CSt-C (50). CD133⁺ cells from brain, prostate and colon cancers are considered CSt-C (53-56). To address the hypothesis that anticancer drugs increase the populations with CSt-C phenotype, we examined expression of these markers on PC lines cultured in the presence or absence of GEM. Table 1.B and FIGS. 6 and 7A,B show that expression of ESA was high in the majority of cancer lines excepting MIA-PaCa-2 and PANC-1. ESA⁺ cells increased in GEM^{Res} cells. The ESA⁺ CD44^{low} CD24^{low} population increased in all GEM^{Res} cells excepting AsPC-1.

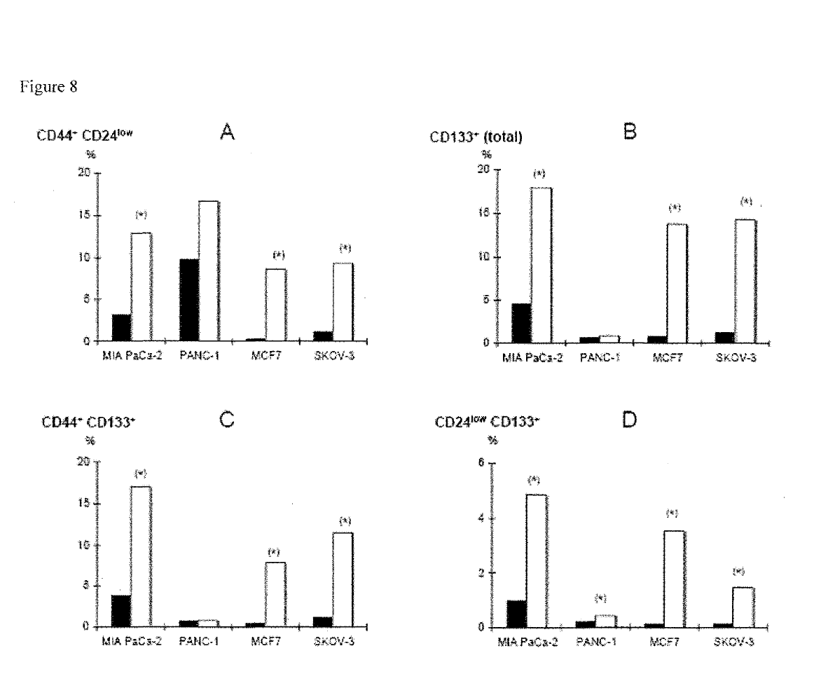

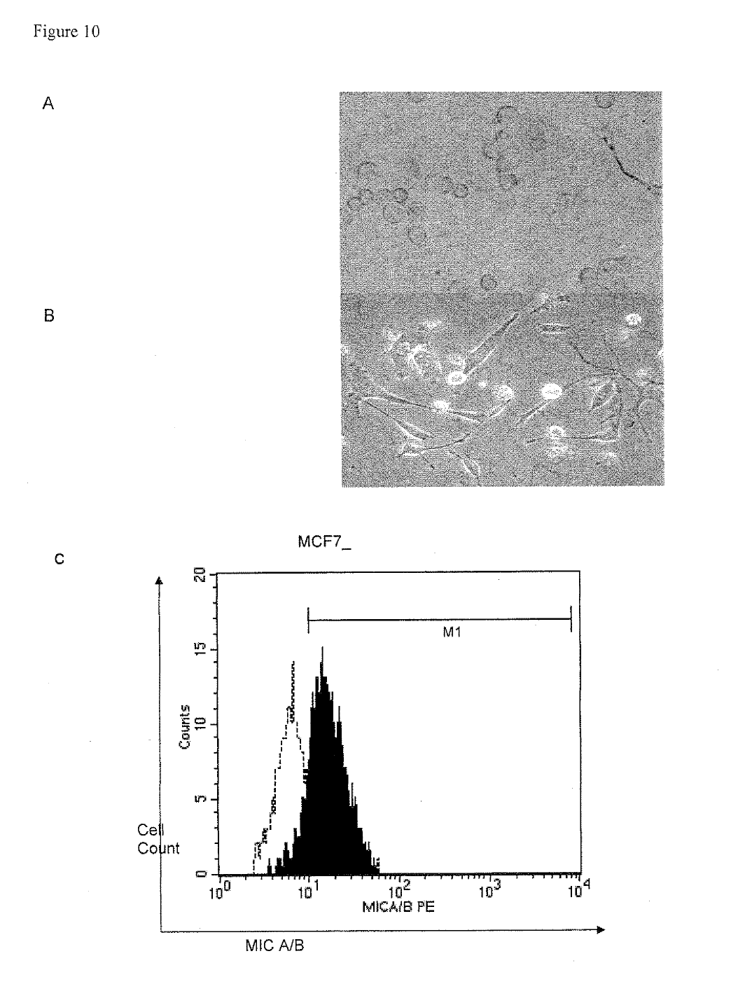

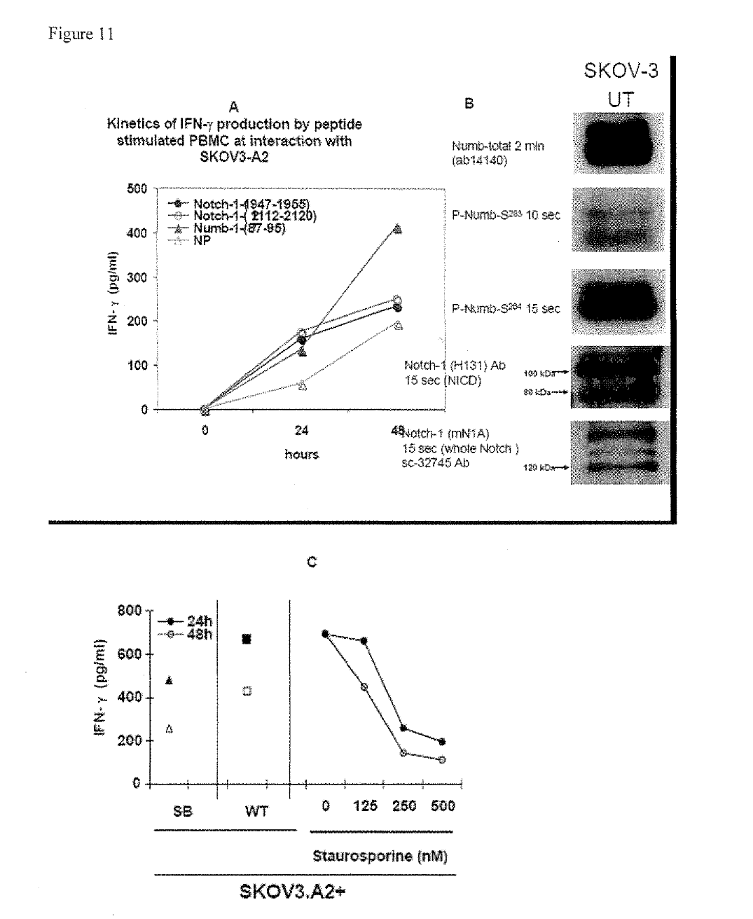

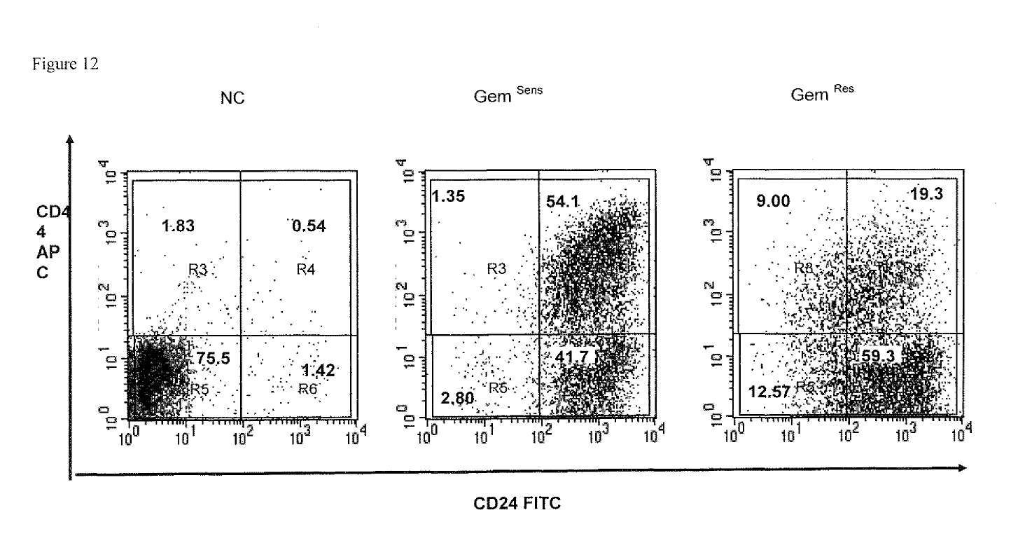

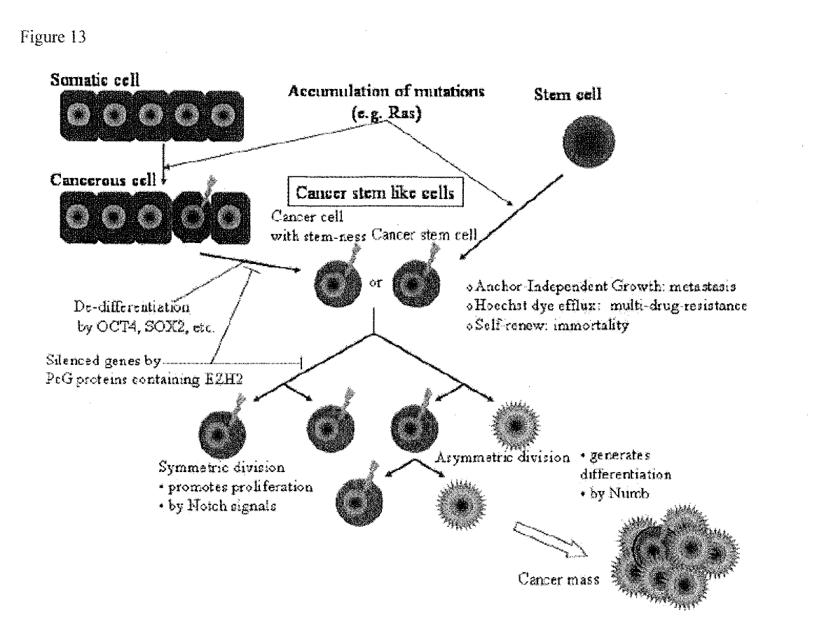

The ESA⁺ CD44^{hi} CD24^{low} and CD133⁺ populations increased in the GEM^{Res} population by 3-5 fold compared with the entire population in Mia-PaCa-2, PANC-1, MCF7 and SKOV3, but not in AsPC-1. (FIG. 8A) The morphologic appearance of live MIA-PaCa-2 cells cultured with GEM changed from round into spindle-shaped or tentaculated cells (FIG. 10A, B). Their appearance was similar with a form of human pancreatic stem cell (57).

Since the ESA⁺ CD44^{hi} CD24^{low} population increased in GEM^{Res} Mia-PaCa-2 and MCF7 we investigated whether other chemotherapeutic drugs had similar effects. CSt-C population increased in MIA-PaCa-2 treated with GEM and 5-FU but not PTX. (FIG. 7A.) For example, starting from 3.0×10⁶ Mia-PaCa-2 cells, 1.3, 3.3, 3.4 and 8.1×10⁶ cells were harvested with GEM, PTX, 5-FU, and without drugs, respectively. 0.6, 0.4, 1.6 and 8.7×10⁶ MCF-7 were harvested after culture of 3×10⁶ MCF-7 cells with GEM, PTX, FU, and no anticancer drug, respectively. GEM and 5-FU increased the CSt-like-C population in both MCF7 and Mia-PaCa-2 while PTX increased that in MCF7. (FIG. 7B).

Chemotherapeutic drugs increase the population expressing the NKG2D ligands in drug-resistant cells.

To address the hypothesis that drug-resistant cancer cells are more sensitive to cellular immune effectors, we quantified expression of NKG2D ligands, MIC-A and -B (58, 59). ESA⁺ MIA-PaCa-2 cells were analyzed for MIC-A/B. MCF7 cells were analyzed with CD44, CD24 and MIC-A/B (FIG. 7B), because almost all MCF7 cells (95% and more) expressed ESA.

MIC-A/B was present on 28.9% of untreated MIA-PaCa-2. GEM^{Res} and 5-FU^{Res} Mia-PaCa-2 cells significantly increased expression of MIC-A/B by more than 3-fold (FIG. 7A). Most ESA⁺ MIA-PaCa-2 cells abundantly expressed MIC-A/B. CSt-like-C increased in entire population of MCF7 resistant to every anticancer drug. However expression of MIC-A/B did not correlate with expression of CD44 and CD24.

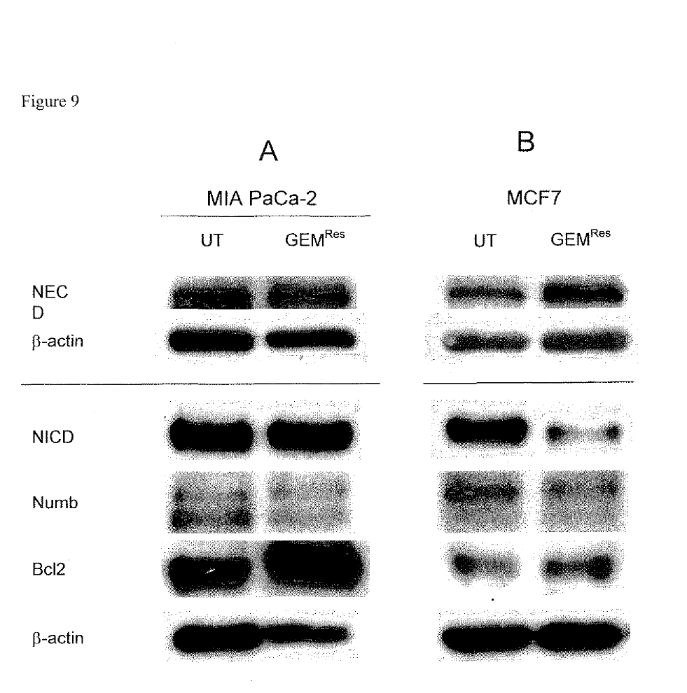

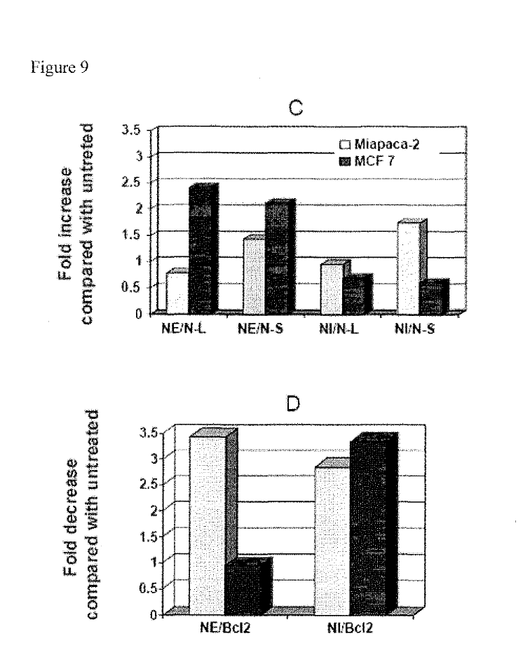

Gemcitibine positively selects MCF7 cells with higher NECD and MIA-PaCa-2 with higher NICD. Notch signals promote survival and proliferation of normal stem cells. Notch signals are mediated by truncated intracellular domain (NICD), which activate transcription in the nucleus. Numb antagonizes Notch signal by inducing degradation of Notch (60, 13). Mammalian Numb has four splicing isoforms, which are divided into two types (Numb^{L} and Numb^{S}) based on the presence or absence of a 49 amino acid insert (5 kDa) in the proline-rich region (PRR) in the C-terminus. It is unclear whether Numb^{L} or Numb^{S} is a significant antagonist of Notch. To characterize expression of Notch and Numb proteins we performed quantitative immunoblot analysis of proteins in the lysates of live MIA-PaCa-2 and MCF7 cultured with or without GEM. (FIG. 9).

Compared with GEM^{Sens} cells, Notch extracellular domain (NECD) expression increased by 18% in GEM^{Res} MIA-PaCa-2, and by 73% in MCF7. In contrast NICD levels slightly increased in MIA-PaCa-2 (by 35%) but decreased by 39% in MCF7. Numb^{L} expression increased by 50% in GEM^{Res} MIA-PaCa-2 but decreased by 29% in GEM^{Res} MCF7. In contrast Numb^{S} decreased by 18% in both GEM^{Res} MIA-PaCa-2 and MCF7. Results indicate that GEM^{Res} MIA-PaCa-2 cells significantly increased the amount of functional NICD, while MCF7 increased NECD with simultaneous decrease in Numb^{L}. Our results indicate that the sensitivity of GEM^{Res} MCF7 to Notch ligands is higher than that of GEM^{Res} MIA-PaCa-2.

Activation of Notch signaling by DLL4 in GEM^{Res} increases CSt-C. Delta-like protein 4 (DLL4) is an endothelial activating ligand of Notch receptor (61, 62). Most (>90%) of GEM^{Res} MCF7 cells were into G1 (resting) phase. Their actual cell number decreased over time. We activated Notch signaling in GEM^{Res} MCF7 with soluble DLL4. DLL4 activated proliferation in the absence and presence of GEM. DLL4+GEM selectively expanded by almost three fold the CSt-C population compared with DLL4 alone (Table 1C). A large number of DLL4-expanded cells were of CD44^{low} CD24^{lo} and CD24^{hi} phenotype. (FIG. 8B). Such cells have been described to be of high metastatic potential since they adhere poorly (63).

Notch and Numb-peptide activated PBMC eliminate CD44^{hi} CD24^{low} and Notch⁺ cells. The finding that MCF7 expresses MIC-A/B, Notch, and Numb proteins, raised the question whether MCF7 are sensitive to IL-2 activated peripheral blood mononuclear cells (PBMC) and Notch and Numb peptide-activated PBMC. Data (not shown) indicates that immunoselection with IL-2-activated PBMC from a healthy HLA-A2-matched donor with MCF7 decreased the number of NICD⁺ MCF7 cells by 36%. Notch-1₂₁₁₂₋₂₁₂₀ peptide-activated PBMC decreased the number of NICD⁺ cells by 50%, while Numb₈₇₋₉₅ peptide-stimulated PBMC mediated a similar non-specific effect with IL-2-activated PBMC.

Therefore a part of peptide-activated PMBC recognized peptides from the Nothc-NICD region presented by HLA-2.

To identify whether activated PBMC inhibited expansion of CSt-like-C, we co-cultured GEM^{Res} and GEM^{Sens} MCF7 with the same activated PBMC. Data (not shown) shows that MCF7 cells did not decrease in numbers during co-culture with IL-2-activated and Notch-1₂₁₁₂₋₂₁₂₀+IL-2-activated PBMC. Numb₈₇₋₉₅+IL-2-activated PBMC significantly decreased the number of MCF7 and of CD44^{hi} CD24^{lo} MCF7 by 2.0-fold compared with IL-2-PBMC.

To address whether GEM^{Res} MCF7 were sensitive to the same immune effectors, we repeated the experiment. Data (not shown) shows that GEM^{Res} cells proliferated slowly and increased in number by only 50% in five days. Co-culture with immune effectors completely inhibited MCF7 proliferation. In contrast, CD44^{hi} CD24^{low} cells which proliferated very slowly, they increased from 53,000 to 60,000 cells in the absence of immune effectors significantly decreased in number by more than 2-fold after immunoselection with IL-2-activated and IL-2 plus peptide-activated PBMC, compared with non-selected GEM^{Res} MCF7. There were no significant differences in survival of GEM^{Res} MCF7 after co-culture with IL-2-activated or peptide-activated PBMC.

The results are consistent with increased MIC-A/B expression on GEM^{Res} MCF7. The NKG2D receptor on cellular immune effectors such as activated NK and CTL, amplify the efficiency of tumor elimination by recognition of MIC-A/B (59). However GEM^{Res} cells of both MCF7 and MIA-PaCa-2 increased MIC-A/B expression, natural immunity alone left some cells which does not express it.

Non-specific cellular immunity is effective to GEM^{Res} cells but CSt-like-C may escape because MIC-A/B did not expressed particularly on CSt-like-C. GEM^{Res} cells containing CSt-like-C required Notch signaling to maintain and overcome to G1 arrest. Notch-1₂₁₁₂₋₂₁₂₀ activated PBMC can delete Notch⁺ cells. Our results support the prospect of acquired specific and natural immunotherapy after chemotherapy especially containing GEM against CSt-like-C.

Discussion

We found that several PC lines, MIA-PaCa-2, PANC-1, and ASPC-1 contained significant populations with breast-CSt-C phenotype. In addition, all lines tested contained populations of significant size expressing colon-CSt-C markers. Phenotypic characterization of pancreatic-CSt-like-C was performed in parallel with the positive control breast MCF7. Functional proteins often provide specific characteristics to cancer cells independent of their tissue origin.

AsPC-1, which was the most sensitive to GEM among all cell lines tested contained a large population of BR-CSt-C phenotype (ESA⁺ CD44^{hi} CD24^{low}) and a small population of colon-CSt-C phenotype. The reasons for high number of cells with this phenotype are unknown. It might possible that since AsPC-1 was isolated from ascites, it originated from CSt-C cells, which invaded and floated from retroperitoneal organs into ascites.

Populations with CSt-C phenotype increased in MIA-PaCa-2 by treatment with GEM or 5-FU but not PTX. However populations of CSt-C remained the same in ASPC-1 and did not increase at treatment with GEM. The lack of change did not correlate with the IC₅₀ for GEM. Our results indicated that pancreatic-CSt-C use distinct pathways for maintenance.

GEM and 5-FU are inhibitors of DNA synthesis, which induce a G0/G1 and S phase arrest and trigger apoptosis in tumor cells (64, 65). PTX inhibits cell division by blocking in the G2 and M phase of the cell cycle and stabilize cytoplasmic microtubules. However cancer cells resting in G1 survive GEM and 5-FU because their nucleic acid synthesis is minimal. In contrast, PTX can interfere with the position of the mitotic spindle, resulting in a symmetric cell division. Numb localization produces asymmetric cell division. PTX can stop both symmetric and asymmetric cell divisions in mitotic step of CSt-C. Thereafter, CSt-C survive and start expanding after the drug decays. Notch receptors are activated by transmembrane ligands of three Delta (DLL1, 2 and, 4) and two Serrate (Jagged-1 and 2) ligands (65). Notch activation by DLL4 was recently reported to be significant for activation of angiogenesis (61, 62). Overexpression of Notch antagonizes Numb expression and suppresses Numb function (14). Therefore, DLL4 boosts symmetric cell division and rapid expansion of CSt-like-C.

Which is the role of GEM in this process? GEM and 5-FU are inhibitors of DNA and RNA synthesis which incorporate in newly synthesized strands. GEM and 5-FU did not affect cells in G₁ phase (64, 66). PTX blocks the G2M phase by stabilizing microtubules. Resting cancer cells rest in G1 survive GEM, 5-FU and PTX because their nucleic acid synthesis is minimal. PTX can interfere with the position of the mitotic spindle, resulting in a symmetric cell division (67, 68). Numb localization produces asymmetric cell division (69). Thereafter, CS-C survive and start expanding after the drug decays. Notch receptors apparently transmit distinct signals when activated by Delta-type (DLL1, 2 and, 4) or Serrate-type (Jagged-1 and 2) ligands. It was recently reported that Notch-ligands induce endocytosis of the NECD in the stimulator cell (70). Soluble ligands such as DLL4 used here, following another study, should be less effective in activating proliferation of CS-C (70).

GEM^{Res} MCF7 and MIA-PaCa-2 differed in the density of NECD, NICD and Numb^{L} MCF7 increased the density of NECD more than MIA-PaCa-2. MCF7 decreased NICD while MIA-PaCa-2 increased NICD. It is tempting to propose that MCF7 increase their “readiness” to respond by increasing the density of Notch receptor, while MIA-PaCa-2 retain more NICD in “stand-by” to activate transcription when the drug is removed. The decrease in Numb^{L} is consistent with the “ready to respond hypotheses”. Because CSt-C were in minority (<30%) in GEM^{Res} cells, future studies are needed to identify the mechanisms and pathways of Notch and Numb activation.

We investigated how these cells can be eliminated. Our first significant finding is that GEM^{Res} cells increased expression of NKG2D ligands, MIC-A and B. Increased expression of MIC-A/B should increase cancer cell sensitivity to NK and CTL and cytokine-activated lymphocytes. This finding provides a supporting rationale for recent findings on the effectiveness of tumor antigen vaccines in PC (71).

Our second significant finding is that Notch and Numb themselves can be targeted by CTL which are specific for Notch-NICD and Numb peptides. NICD peptides are generated from degraded NICD after signaling. Numb peptides are generated after Numb phosphorylation. In this scenario the GEM^{Res} tumor becomes a target for CTL when Numb is degraded and CS-C proliferation is activated. Furthermore, NICD becomes a good target for CTL when the cancer cell is in the “ready to respond” state. The observed decrease in Numb in both lines and of NICD in MCF7 suggest that such approach will be effective immediately after chemotherapy. CSt-C were recently reported to be resistant to radiation (72) and chemotherapy (this study). Infusion of patients with advanced pancreatic cancer with autologous, tumor-antigen activated T and NK cells may extend the survival of such patients.

### Example 3

### Cancer-Stem-Cell-Like Cells (CSt-C) in Human Solid Tumors

A stem cell (St-C) is a cell which has the ability both to self-renew and to differentiate multidirectionally. Stem cells are required during generation and early development of organs but also during repairing and maintenance of injured or immflammational damage of various tissues.

Mutations in some genes e.g. RAS are sufficient to endow a cell with a full cancer phenotype. Cancer stem cells (C-St-Cs) result from accumulation of mutations in proto-oncogenes. C-St-Cs represent biologically distinct clones that are capable of self-renewal and sustaining tumor growth in vivo with ability of self-renewal differentiation. C-St-Cs were identified in hematopoietic cancers and solid tumors such as breast, brain, prostate, and colon cancer. C-St-Cs possess almost all of typical malignant characteristics, such as radiation- and multidrug-resistance and anchor-independent growth. Thus, classical treatment modalities rather create nutrient-rich niches for C-St-Cs, than eliminate these cells. New strategies of molecular targeting therapy are needed. In this example, we focus on the appropriate targets for elimination of C-St-Cs.

Symmetric/Asymmetric Division of Stem Cell and Cancer Development

A St-C has two types of division, symmetric and asymmetric. Symmetric cell division of parent St-C-yields two daughter St-C with the same ability of parent St-C and increase St-C numbers. Asymmetric cell division generates one identical daughter (self-renewal) and one daughter that differentiates. Asymmetric division is regulated by intracellular and extracellular mechanisms. The first determine the asymmetric partitioning of cell components that determine cell fate. External factors mediate the asymmetric placement of daughter cells relative to microenvironment (St-C niche and exposure to signals).

Symmetric St-C divisions observed during the development are also common during wound healing and regeneration. St-C undergo symmetric divisions to expand St-C pools of undifferentiated daughter cells during embryonic or early fetal development. Symmetric St-C divisions were also observed in adults. In the Drosophila ovary, adult germline stem cells divide asymmetrically, retaining one daughter with the stem cell fate in the niche and placing the other outside the niche to differentiate. However, female germline St-C can be induced to divide symmetrically and to regenerate an additional St-C after experimental manipulation, in which, one St-C is removed from the niche.

Mammalian stem cells also switch between symmetric and asymmetric cell divisions. Both neural and epidermal progenitors change from mainly symmetric divisions that expand St-C pools during embryonic development to mainly asymmetric divisions that expand differentiated cell numbers in mid to late gestation. Symmetric St-C self-renewal and expansion confer developmental plasticity, increased growth and enhanced regeneration. However, St-C self-renewal also contains an inherent risk of cancer. Drosophila neuroblasts divide asymmetrically as a result of the asymmetric localization of: (i) cortical cell polarity determinants (such as Partner of Inscuteable (PINS) and an atypical protein kinase C (a-PKC)), (ii) cell fate determinants (e.g. Numb and Prospero), and (iii) regulated alignment of the mitotic spindle. When the machinery that regulates asymmetric divisions is disrupted, neuroblasts divide symmetrically and form tumors.

Cell clones lacking PINS are tumorigenic. Double mutant cells lacking both PINS and Lethal giant larvae (LGL) generate a brain composed largely of symmetrically dividing and self-renewing neuroblasts. Cell clones lacking the cell fate determinants Numb or Prospero are also tumorigenic and can be propagated after transplantation into new hosts. These tumor cells have been shown to become aneuploid within 40 days of adopting a symmetric mode of division. Therefore, the capacity to divide symmetrically may be a prerequisite for neoplastic transformation. Cancer may reflect, at least in part, the capacity to adopt a symmetric mode of cell division.

The machinery that promotes asymmetric cell divisions has an evolutionarily conserved role in tumor suppression. The adenomatous polyposis coli (APC) gene is required for the asymmetric division of Drosophila spermatogonial stem cells and is an important tumor suppressor in the mammalian intestinal epithelium. It is not known whether APC regulates asymmetric division by St-C in the intestinal epithelium, but colorectal cancer cells have properties that are strikingly similar to those of intestinal epithelial St-C. The human homologue of LGL, HUGL-1, is also frequently deleted in cancer, and deletion of the corresponding gene in mice leads to a loss of polarity and dysplasia in the central nervous system. Loss of Numb may be involved in the hyperactivation of Notch pathway signaling observed in breast cancers. Although these gene products could inhibit tumorigenesis through various mechanisms that are independent of their effects on cell polarity, the fact that these genes consistently function as tumor suppressors suggests that asymmetric division itself may protect against cancer.

Further evidence for the link between symmetric cell divisions and cancer is the observation that some gene products can both induce symmetric cell divisions and function as oncogenes in mammalian cells. aPKC normally localizes to the apical cortex of the neuroblast as part of the PAR3/6-aPKC complex. Neural-specific expression of a constitutively active variant of aPKC causes a large increase in symmetrically dividing neuroblasts. Consistent with this tumorigenic potential in Drosophila, aPKC has been also identified as an oncogene in human lung cancers. Thus, asymmetric division may suppress carcinogenesis. Regulation of St-C to switch to asymmetric division may suppress cancer progression.

Notch and Numb Play Important Roles in Symmetric/Asymmetric Division

Notch encodes a transmembrane receptor that after cleavage release an intracellular domain (NICD) that is directly involved in transcriptional activation in the nucleus. Notch activation promotes the survival of neural St-C by induction of the expression of its specific target genes: hairy and enhancer of split 3 (Hes3) and Sonic hedgehog (Shh) through rapid activation of cytoplasmic signals. The Notch ligand, Delta-like 4 (DLL4) rapidly inhibit cell death. Cells exposed to Notch ligands retain the potential to generate neurons, astrocytes and oligodendrocytes after prolonged exposure to Notch ligands. Cells stimulated to divide by DLL4 survive for long periods in the parenchyma of the normal brain in an immature state, suggesting upregulation of pro-survival molecules.

The Notch antagonist Numb decreases the amount of Notch and in that modifies the response of daughter cells to Notch signals of the (Notch^{hi} cells can both receive and transmit signals to neighbouring cells, while Notch^{lo} cells can only receive Notch signals. Inhibition of Notch signaling by Numb seems to be involved in the regulation of mammalian asymmetric division. Undifferentiated neural progenitors in the developing rodent cortex distribute Numb asymmetrically to precursors destined for neurogenesis. Thus, asymmetric segregation of Numb in myocytes may be a common mode of control. During delaminating from the asymmetric division of a neuroblast, Numb and several other proteins are co-localized in a basal cortical crescent as intrinsic determinants. These proteins are partitioned to the basal daughter cell or the ganglion mother cell, which will divide once more, generating two neurons or a neuron and a glial cell. The apical daughter to which the proteins were not partitioned maintains the neuroblast characteristics and is capable of undergoing several additional rounds of cell division.

The N-terminal phosphotyrosine-binding (PTB) domain, recruits Numb to the membrane. Numb-PTB domain interacts specifically with NIP (Numb-interacting protein), which is an intrinsic membrane protein that recruits Numb from the cytosol to the plasma membrane. Numb-PTB domain also can interact with LNX (ligand of Numb X) which acts as an E3 ligase for the ubiquitination and degradation of mNumb Mammalian Numb (mNumb) has four splicing isoforms. They are divided by into two types based on the presence or absence of a 50 amino acid insert in proline-rich region (PRR) in the C-terminus. The human isoforms with a long PRR domain (Numb-PRR^{L}) promote proliferation of cells without affecting differentiation during early neurogenesis in central nervous system (CNS). The. isoforms with a short PRR domain (Numb-PRR^{S}) inhibit proliferation of the stem cells and promote neuronal differentiation. Numb-PRR^{S} decreases the amount of Notch and antagonizes the activity of Notch signaling stronger than Numb-L. In contrast, negative regulation ubiquitination of Numb targets the PTB^{L} variants which contain a charged decapeptide.

We found distinct levels of expression of Numb L and Numb S in breast MCF-7 pancreas Miapaca-2 and ovarian SKOV3 lines. Expression of Numb might be an indicator of the symmetric/asymmetric division potential of C-St-C and its relation to cancer activitivation. Further studies are needed to address this question.

Polycomb Group Proteins Target Genes that Pluripotent Factors Target

Polycomb group (PcG) proteins are transcriptional repressors that maintain cellular identity during metazoan development through epigenetic modification of chromatin structure. PcG proteins transcriptionally repress developmental genes in embryonic stem cells (E-St-C), the expression of which would otherwise promote differentiation. PcG-bound chromatin is trimethylated at Lys27 (K²⁷) of histone-H3 and is transcriptionally silent. The Octamer-binding transcription factor-4 (OCT4), the SRY-related high-mobility group (HMG)-box protein-2 (SOX2), and the Homeodomain-containing transcription factor, NANOG, genes are PcG targets, indicating that chromatin modifiers might act in concert with these three pluripotency regulators to directly repress developmental pathways in ESf-C cells. OCT4 is expressed in adult pluripotent St-C and several human and rat tumor cells, but not in normal differentiated daughters of these St-C. Adult cells expressing the Oct4 gene are potential pluripotent St-C and relative with initiation of the carcinogenic process. SOX2. is implicated in the regulation of transcription and chromatin architecture. SOX2 participates in the regulation of the inner cell mass (ICM) and its progeny or derivative cells by forming a ternary complex with either OCT4 or the ubiquitous OCT1 protein on the enhancer DNA sequences of fibroblast-growth factor-4 (Fgf4). Nanog confers leukemia inhibitory factor (LIF)-independent ability for cell renewal and pluripotency of mouse Est-C. Nanog was first described as ENK (early embryo-specific NK) due to its homology with members of the NK gene family. Nanog mRNA is present in primordial germ and embryonic germ cells. Nanog protein was not found in Stella-positive mouse primordial germ cells, despite Stella itself being considered a marker of pluripotency. The function of Nanog in germ cells is progressively extinguished as they mature. Nanog might repress transcription of genes that promote differentiation.

The chromatin conformation associated with many developmental genes is composed of “pivalent domains” consisting of both inhibitory methylated K²⁷ and activating methylated K⁴ histone in H-3. These bivalent domains are lost in differentiated cells, suggesting that they play an important part in maintaining developmental plasticity of ES cells. Thus, OCT4, SOX2 and NANOG might act in concert with PcG proteins to silence key developmental regulators in the pluripotent state.

Gene inactivation by PcG requires cooperation of two complexes of the various PcG proteins: (i) Polycomb repressive complex 1 (PRC1) binds to chromatin, and blocks the effects of a known gene-activating protein complex, and (ii) PRC2 leads PRC 1 to target genes. One of PRC2 components, known as E(Z) for Enhancer of Zeste, has the ability to add methyl (CH3) groups to K²⁷, which is located in the tail at the end of H-3 of chromatin. The histone modifications play a major role in regulating the activity of genes, turning them either on or off, depending on the modification. In PRC2 case, CH3 addition turns genes off, by attracting PRC1 to the genes to be inactivated. The PRC2's methylating activity is needed for PRC 1 binding.

Expression of EZH2, the human equivalent of the fruit fly E(z) protein, is much higher in metastases of prostate and breast cancers than it is in localized tumors or normal tissue. Expression of EZH2 in cancer tissues was reported to correlate with poor prognosis and malignant potential such as high proliferation, spreading and invasion of melanoma, breast, prostate, endometrium and stomach cancers. Blocking production of the E(Z) protein inhibited proliferation of prostate cancer cells. EZH2 may inhibit tumor-suppressor genes or genes that make proteins that keep cells anchored in place. EZH2 overexpression and formation of the PRC variant occurs in undifferentiated cells as well as in cancer cells. The histone methylation mediated by EZH2 helps maintain stem cells in their pluripotent developmental state.

Cancer Might be Caused from Cancer-Stem-Like Cell Obtained by De-Differentiation

1) Pluripotent factors are required to make stem-like cells from mature cells.

Some cancers could be caused from de-differentiated cancer cells with stem-cell-ness. In addition to OCT4, SOX2, and Nanog, c-myc and Klf4 also contribute to the long-term maintenance of the Est-C phenotype and the rapid proliferation of Est-C in culture. Induction of pluripotent stem cells from adult mouse fibroblasts was demonstrated by introducing, Oct4, Sox2, c-Myc and Klf4, suggesting that mature cell can revert into immature under special circumstance, and then some cancer cells might obtain stem-cell-ness. How these factors affect each other? Increased expression of Oct4 causes mouse Est-C to differentiate into extra-embryonic endoderm and mesoderm, whereas increased expression of Nanog enhances self-renewal and maintenance of the undifferentiated state. Decreased expression of Oct4 causes mouse Est-C to differentiate into trophectoderm. This indicates that Oct4 and Nanog operate independently and their primary function might be the repression of embryonic-cell differentiation. A combined signal from both proteins leads to renewal and pluripotency of the primitive ectoderm. The octamer and sox elements are required for the upregulation of mouse and human Nanog transcription. OCT4, SOX2 and Nanog cooperate with additional transcription factors. They are essential but not sufficient for specification of a pluripotent cellular state. Characterization of the upstream control of Oct4 and Nanog expression is very important.

2) Cancer Cells Might Obtain Stem-Cell-Ness.

Cancer cells have malignant potential usually defined long survival, distant metastases, and anticancer-drug resistance. C-St-Cs were reported in breast, brain, prostate and colon. Since breast, pancreatic and ovarian cancers are of epithelial origin, they express the epithelial marker ESA. Some but all pancreatic cancer (PC) cell lines tested expressed the CSt-C characteristic phenotype: CD44⁺ CD24^{low/-}. Surprisingly, the ESA⁺ CD44⁺CD24^{low/-} population increased after culture with gemcitabine (GEM) or 5-fluorouracil (FU). The DNA and RNA synthesis inhibitors GEM and 5-FU are among the most effective anti-cancer drugs. Positive selection of C-St-Cs by drugs and radiation lends support to two hypotheses. The first is that C-St-Cs are enriched in the resistant population because they express high levels of anti-apoptotic molecules and are simultaneously in G−1 resting state. The second is that resistant cells divide slowly and “asymmetrically” after changing the position of the mitotic spindle, i.e., de-differentiation. These hypotheses are summarized in FIG. 13.

Elimination of C-St-C

All studies concur that C-St-C are resistant to chemotherapy and radiotherapy. The first approach to eliminate C-St-C is to negatively regulate the genetic pathways which promote symmetric cell division. The function of all genes and proteins listed above can be negatively regulated by antagonistic gene-products.

One possibility consists in expression of antagonists of Notch in cancer cells (FIG. 2). mRNA encoding for Numb or its PTB-domain can be expressed in tumor cells from a negative strand RNA vector. Such vectors are based on Newcastle disease virus or Sendai virus. Unfortunately, recent concerns about bird flu limit the attractivity of this approach.

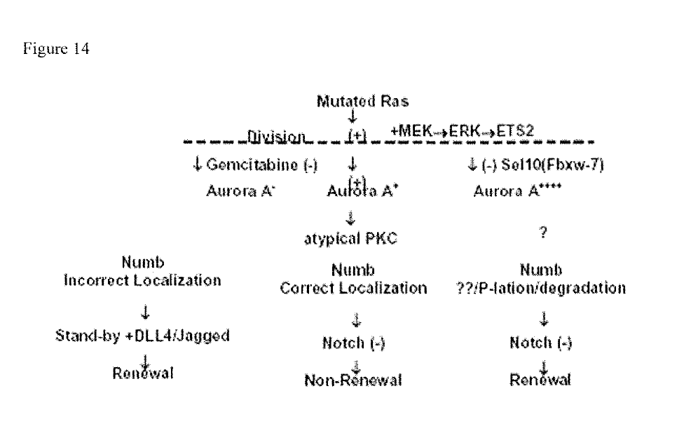

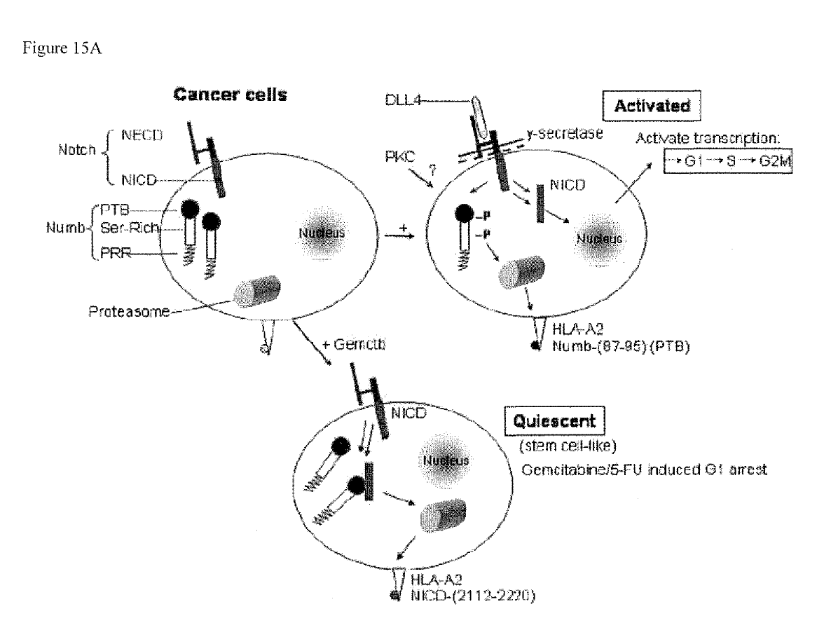

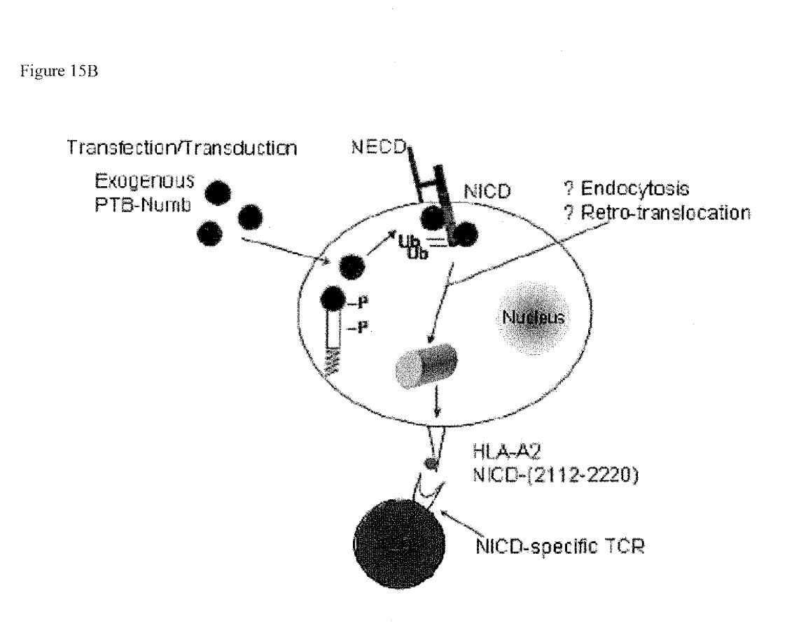

The alternative is degradation of proteins which positively control activation pathways. Mammalian Aurora-A has been termed an oncogene due to its overexpression in several cancers, its ability to promote proliferation in certain cell lines and the fact that reduced levels lead to multiple centrosomes, mitotic delay and apoptosis. A proposed mechanism is described below. Aurora-A is overexpressed in PC lines including MIA-PaCa-2, is activated by the pathway: MAPK-ERK-ETS2. It is unclear how mammalian Aurora-A regulates stem cell asymmetric division and self-renewal, it is involved in PC oncogenesis and cooperates with Ras- or Myc-signals. A recent study finds that the decreases in the UB-ligase E3 Sel10, allows prolonged and sustained Aurora-A signals, whose targets promote self-renewal of cancer cells. Expression of Ub-ligases in cancer cells may be helpful. See FIG. 14.

The second approach is to develop more specific small molecule inhibitors of PKC and aPKC to inhibit asymmetric division. Such inhibitors are important in a different context. Taxol affects polymerization of microtubules. It is possible that some of taxol-resistant cells re-position the mitotic spindle. Ovarian and PC treated with taxol increased the number of CD44⁺ CD24^{lo} cells.

A third approach results from apparently unrelated studies. The EZH2 protein was targeted by active specific tumor immunotherapy. CTL recognizing peptide sequences of EZH2 restricted by HLA-A24 manner were identified. A vaccine trial with EZH2 is ongoing in patients with prostate and brain cancer. The question is whether high expression of EZH2 results in high turnover rate. Only in this scenario EZH2 focussed immunotherapy will eliminate CSt-C. See FIGS. 17A-17B.

We believe that Numb and Notch themselves are appropriate targets for elimination of Cst-C by activated CTL. Cst-C, which activate proliferation by Notch ligands degrade Numb and present. Numb peptides bound to HLA-A,B,C. These complexes can be recognized by Numb peptide-specific CTL and eliminated. Alternatively, CSt-C in resting state degrade Notch. Notch peptides-HLA, ABC complexes presented by tumors transform Cst-C in targets for Notch peptide specific CTL.

Conclusion

Proliferation and differentiation of St-C defined as abilities of both self-renewal and pluriotency, are regulated by symmetric/asymmetric cell divisions. Notch signaling pathways balance these divisions. Numb plays an important role in stem cell divisions, not only through repression of Notch signaling but also through its isoforms as intrinsic predictive determinant. Expression of Notch and Numb might indicate the metastatic potential of CSt-C. Anticancer drug select or induce CSt-C. CST-C require pluripotent factors and PcG proteins to maintain and expand. Therefore, Numb, Notch, PKC, aPKC and EZH2 should be appropriate targets for St-C elimination following chemotherapy and radiotherapy.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Artavanis-Tsakonas S, Rand M D and Lake R J: Notch signaling: cell fate control and signal integration in development. Science 284: 770-6, 1999.
2. Mumm J S, Schroeter E H and Saxena M T et al: A ligand-induced extracellular cleavage regulates gamma-secretase-like proteolytic activation of Notch1. Mol. Cell 5: 197-206, 2000.
3. Ellisen L W, Bird J and West D C et al: TAN-1, the human homolog of the Drosophila notch gene, is broken by chromosomal translocations in T lymphoblastic neoplasms. Cell 66: 649-61, 1991.
4. Weng A P, Ferrando A A and Lee W et al: Activating mutations of NOTCH1 in human T cell acute lymphoblastic leukemia. Science 306: 269-71, 2004.
5. Radtke F and Raj K: The role of Notch in tumorigenesis: oncogene or tumour suppressor? Nat. Rev. Cancer. 3: 756-67, 2003.
6. Leong K G and Karsan A: Recent insights into the role of Notch signaling in tumorigenesis. Blood 107: 2223-33, 2006.
7. Capobianco A J, Zagouras P, Blaumueller C M, Artavanis-Tsakonas S and Bishop J M: Neoplastic transformation by truncated alleles of human NOTCH1/TAN1 and NOTCH2. Mol. Cell. Biol. 17: 6265-73, 1997.
8. Nickoloff B J, Osborne B A and Miele L: Notch signaling as a therapeutic target in cancer: a new approach to the development of cell fate modifying agents. Oncogene 22; 6598-608, 2003.
9. Reya T, Morrison S J, Clarke M F and Weissman I L: Stem cells, cancer, and cancer stem cells. Nature 414: 105-11, 2001.
10. Martinez Arias A, Zecchini V and Brennan K: CSL-independent Notch signalling: a checkpoint in cell fate decisions during development? Curr. Opin. Genet. Dev. 12: 524-33, 2002.
11. Guo M, Jan L Y and Jan Y N: Control of daughter cell fates during asymmetric division: interaction of Numb and Notch. Neuron 17: 27-41, 1996.
12. Rhyu M S, Jan L Y and Jan Y N: Asymmetric distribution of numb protein during division of the sensory organ precursor cell confers distinct fates to daughter cells. Cell 76: 477-91, 1994.
13. Santolini E, Puri C and Salcini A E et al: Numb is an endocytic protein. J. Cell Biol. 151: 1345-52, 2000.
14. Pece S, Serresi M and Santolini E et al: Loss of negative regulation by Numb over Notch is relevant to human breast carcinogenesis. J. Cell Biol. 167: 215-21, 2004.
15. Stylianou S, Clarke R B and Brennan K: Aberrant activation of notch signaling in human breast cancer. Cancer Res. 66: 1517-25, 2006.
16. Gomez-Nunez M, Pinilla-Ibarz J and Dao T et al: Peptide binding motif predictive algorithms correspond with experimental binding of leukemia vaccine candidate peptides to HLA-A*0201 molecules. Leuk. Res. 2006.
17. Bian H, Reidhaar-Olson J F and Hammer J: The use of bioinformatics for identifying class II-restricted T-cell epitopes. Methods 29: 299-309, 2003.
18. Nussbaum A K, Kuttler C, Hadeler K P, Rammensee H G and Schild H: P AProC: a prediction algorithm for proteasomal cleavages available on the WWW. Immunogenetics 53: 87-94, 2001.
19. Kopp J and Schwede T: The SWISS-MODEL Repository: new features and functionalities. Nucleic Acids Res. 34: 0315-8, 2006.
20. Zwahlen C, Li S C, Kay L E, Pawson T and Forman-Kay J D: Multiple modes of peptide recognition by the PTB domain of the cell fate determinant Numb. EMBO J. 19: 1505-15, 2000.
21. Ehebauer M T, Chirgadze D Y, Hayward P, Martinez Arias A and Blundell T L: High-resolution crystal structure of the human Notch1 ankyrin domain. Biochem. J. 392: 13-20, 2005.
22. Hambleton S, Yaleyev N Y and Muranyi A et al: Structural and functional properties of the human notch-1 ligand binding region. Structure 12: 2173-83, 2004.
23. Weijzen S, Rizzo P and Braid M et al: Activation of Notch-1 signaling maintains the neoplastic phenotype in human Ras-transformed cells. Nat. Med. 8: 979-86, 2002.
24. Fisk B, Blevins T L, Wharton J T and Ioannides C G: Identification of an immunodominant peptide of HER-2/neuprotooncogene recognized by ovarian tumor-specific cytotoxic T lymphocyte lines. J. Exp. Med. 181: 2109-17, 1995.
25. Frischmann U and Muller W: Nine fluorescence parameter analysis on a four-color fluorescence activated flow cytometer. Cytometry A. 69: 124-6, 2006.
26. Kawano K, Efferson C L and Peoples G E et al: Sensitivity of undifferentiated, high-TCR density CD8⁺ cells to methylene groups appended to tumor antigen determines their differentiation or death. Cancer Res. 65: 2930-7, 2005.
27. Fahmy T M, Bieler J G, Edidin M and Schneck J P: Increased TCR avidity after T cell activation: a mechanism for sensing low-density antigen. Immunity 14: 135-43, 2001.
28. Fahmy T M, Bieler J G and Schneck J P: Probing T cell membrane organization using dimeric MHC-Ig complexes. J. Immunol. Methods 268: 93-106, 2002.
29. Frahm N, Korber B T and Adams C M et al: Consistent cytotoxic-T-lymphocyte targeting of immunodominant regions in human immunodeficiency virus across multiple ethnicities. J. Virol. 78: 2187-200, 2004.
30. Ellis J M, Henson V, Slack R, Ng J, Hartzman R J and Katovich Hurley C: Frequencies of HLA-A2 alleles in five U.S. population groups. Predominance Of A*02011 and identification of HLA-A*0231. Hum. Immunol. 61: 334-40, 2000.
31. Li J L and Harris A L: Notch signaling from tumor cells: a new mechanism of angiogenesis. Cancer. Cell. 8: 1-3, 2005.
32. Rehman A O and Wang C Y: Notch signaling in the regulation of tumor angiogenesis. Trends Cell Biol. 16: 293-300, 2006.
33. Patel N S, Li J L, Generali D, Poulsom R, Cranston D W and Harris A L: Up-regulation of delta-like 4 ligand in human tumor vasculature and the role of basal expression in endothelial cell function. Cancer Res. 65: 8690-7, 2005.
34. Mailhos C, Modlich U, Lewis J, Harris A, Bicknell Rand Ish-Horowicz D: Delta4, an endothelial specific notch ligand expressed at sites of physiological and tumor angiogenesis. Differentiation 69: 135-44, 2001.
35. Hainaud P, Contreres J O and Villemain A et al: The Role of the Vascular Endothelial Growth Factor-Delta-like 4 Ligand/Notch4-Ephrin B2 Cascade in Tumor Vessel Remodeling and Endothelial Cell Functions. Cancer Res. 66: 8501-10, 2006.
36. Hopfer O, Zwahlen D, Fey M F and Aebi S: The Notch pathway in ovarian carcinomas and adenomas. Br. J. Cancer 93: 709-18, 2005.
37. Park J T, Li M and Nakayama K et al: Notch3 gene amplification in ovarian cancer. Cancer Res. 66: 6312-8, 2006.
38. Sette A and Sidney J: Nine major HLA class I supertypes account for the vast preponderance of HLA-A and -B polymorphism. Immunogenetics 50: 201-12, 1999.
39. Parkin D M, Bray F, Ferlay J and Pisani P: Global cancer statistics, 2002. CA Cancer. J. Clin. 55: 74-108, 2005.
40. Androutsellis-Theotokis A, Leker R R, Soldner F, et al. Notch signalling regulates stem cell numbers in vitro and in vivo. Nature 2006; 442:823-6.
41. Bao S, Wu Q, McLendon R E, et al. Glioma stem cells promote radioresistance by preferential activation of the DNA damage response. Nature 2006; 444:756-60.
42. Morrison S J, Kimble J. Asymmetric and symmetric stem-cell divisions in development and cancer. Nature 2006; 441:1068-74.
43. Rothenberg M L, Moore M J, Cripps M C, et al. A phase II trial of gemcitabine in patients with 5-FU-refractory pancreas cancer. Ann Oncol 1996; 7:347-53.
44. Burris H A 3rd, Moore M J, Andersen J, et al. Improvements in survival and clinical benefit with gemcitabine as first-line therapy for patients with advanced pancreas cancer: a randomized trial. J Clin Oncol 1997; 15:2403-13.
45. Berlin J D, Catalano P, Thomas J P, Kugler J W, Haller D G, Benson A B 3rd. Phase III study of gemcitabine in combination with fluorouracil versus gemcitabine alone in patients with advanced pancreatic carcinoma: Eastern Cooperative Oncology Group Trial E2297. J Clin Oncol 2002; 20:3270-5.
46. Rocha Lima C M, Green M R, Rotche R, et al. Irinotecan plus gemcitabine results in no survival advantage compared with gemcitabine monotherapy in patients with locally advanced or metastatic pancreatic cancer despite increased tumor response rate. J Clin Oncol 2004; 22:3776-83.
47. Van Cutsem E, van de Velde H, Karasek P, et al. Phase III trial of gemcitabine plus tipifarnib compared with gemcitabine plus placebo in advanced pancreatic cancer. J Clin Oncol 2004; 22:1430-8.
48. Bramhall S R, Rosemurgy A, Brown P D, Bowry C, Buckels J A. Marimastat Pancreatic Cancer Study Group: Marimastat as first-line therapy for patients with unresectable pancreatic cancer: a randomized trial. J Clin Oncol 2001; 19:3447-55.
49. Moore M J, Hamm J, Dancey J, et al. Comparison of gemcitabine versus the matrix metalloproteinase inhibitor BAY 12-9566 in patients with advanced or metastatic adenocarcinoma of the pancreas: a phase III trial of the National Cancer Institute of Canada Clinical Trials Group. J Clin Oncol 2003; 21:3296-302.
50. Al-Hajj M, Wicha M S, Benito-Hernandez A, Morrison S J, Clarke M F. Prospective identification of tumorigenic breast cancer cells. Proc Natl Acad Sci USA 2003; 100:3983-8.
51. Li C, Heidt D G, Dalerba P, et al. Identification of pancreatic cancer stem cells. Cancer Res 2007; 67:1030-7.
52. Krause D S, Lazarides K, von Andrian U H, Van Etten R A. Requirement for CD44 in homing and engraftment of BCR-ABL-expressing leukemic stem cells. Nat Med 2006; 12:1175-80.
53. Singh S K, Clarke I D, Terasaki M, et al. Identification of a cancer stem cell in human brain tumors. Cancer Res 2003; 63:5821-8.
54. Ricci-Vitiani L, Lombardi D G, Pilozzi E, et al. Identification and expansion of human colon-cancer-initiating cells. Nature 2007; 445:111-5.
55. O′Brien C A, Pollett A, Gallinger S, Dick J E. A human colon cancer cell capable of initiating tumour growth in immunodeficient mice. Nature 2007; 445:106-10.
56. Collins A T, Berry P A, Hyde C, Stower M J, Maitland N J. Prospective identification of tumorigenic prostate cancer stem cells. Cancer Res 205; 65:10946-51.
57. Tai M H, Chang C C, Kiupel M, et al. Oct4 expression in adult human stem cells: evidence in support of the stem cell theory of carcinogenesis. Carcinogenesis 2005; 26:495-502.
58. Gasser S, Orsulic S, Brown E J, Raulet D H. The DNA damage pathway regulates innate immune system ligands of the NKG2D receptor. Nature 2005; 436:1186-90.
59. Skov S, Pedersen M T, Andresen L, Straten P T, Woetmann A, Odum N. Cancer cells become susceptible to natural killer cell killing after exposure to histone deacetylase inhibitors due to glycogen synthase kinase-3-dependent expression of MHC class I-related chain A and B. Cancer Res 2005; 65:11136-45.
60. Berdnik D, Torok T, Gonzalez-gaitan M, Knoblich J A. The endocytic protein alpha-Adaptin is required for numb-mediated asymmetric cell division in Drosophila. Dev Cell 2002; 3:221-31.
61. Noguera-Troise I, Daly C, Papadopoulos N J, et al. Blockade of D114 inhibits tumour growth by promoting non-productive angiogenesis. Nature 2006; 444:1032-7.
62. Ridgway J, Zhang G, Wu Y, et al. Inhibition of D114 signalling inhibits tumour growth by deregulating angiogenesis. Nature 2006; 444:1083-7.
63. Kobayashi H, Boelte K C, Lin P C. Endothelial cell adhesion molecules and cancer progression. Curr Med Chem. 2007; 14(4):377-86
64. Bellone G, Carbone A, Busso V, Scirelli T, Buffolino A, Smirne C, Novarino A, Bertetto O, Tosetti L, Emanuelli G. Antagonistic interactions between gemcitabine and 5-fluorouracil in the human pancreatic carcinoma cell line Capan-2. Cancer Biol Ther. 2006 October; 5(10):1294-303
65. Le Borgne R. Regulation of Notch signalling by endocytosis and endosomal sorting. Curr Opin Cell Biol. 2006, 18(2):213-22.
66. Tolis C, Peters G J, Ferreira C G, Pinedo H M, Giaccone G. Cell cycle disturbances and apoptosis induced by topotecan and gemcitabine on human lung cancer cell lines. Eur J Cancer 1999; 35:796-807.
67. Bowman S K, Neumuller R A, Novatchkova M, Du Q, Knoblich J A. The Drosophila NuMA Homolog Mud regulates spindle orientation in asymmetric cell division. Dev Cell. 2006 June; 10(6):731-42.
68. Betschinger J, Mechtler K, Knoblich J A. Asymmetric segregation of the tumor suppressor brat regulates self-renewal in Drosophila neural stem cells. Cell. 2006 Mar. 24; 124(6):1241-53.
69. Smith C A, Lau K M, Rahmani Z, Dho S E, Brothers G, She Y M, Berry D M, Bonneil E, Thibault P, Schweisguth F, Le Borgne R, McGlade C J. aPKC-mediated phosphorylation regulates asymmetric membrane localization of the cell fate determinant Numb. EMBO J. 2007 24; 26(2):468-80
70. Nichols J T, Miyamoto A, Olsen S L, D'Souza B, Yao C, Weinmaster G. DSL ligand endocytosis physically dissociates Notch1 heterodimers before activating proteolysis can occur. J Cell Biol. 2007 Feb. 12; 176(4):445-58.
71. Yanagimoto H, Mine T, Yamamoto K, Satoi S, Terakawa N, Takahashi K, Nakahara K, Honma S, Tanaka M, Mizoguchi J, Yamada A, Oka M, Kamiyama Y, Itoh K, Takai S. Immunological evaluation of personalized peptide vaccination with gemcitabine for pancreatic cancer. Cancer Sci. 2007 Feb. 12
72. Phillips T M, McBride W H, Pajonk F. The response of CD24(−/low)/CD44+ breast cancer-initiating cells to radiation. J Natl Cancer Inst. 2006 98(24):1777-85.
73. Efferson C L, Tsuda N, Kawano K, Nistal-Villan E, Sellappan S, Yu D, Murray J L, Garcia-Sastre A, Ioannides C G. Prostate tumor cells infected with a recombinant influenza virus expressing a truncated NS1 protein activate cytolytic CD8+ cells to recognize noninfected tumor cells. J. Virol. 2006 January; 80(1):383-94.
74. Castilleja A, Carter D, Efferson C L, Ward N E, Kawano K, Fisk B, Kudelka A P, Gershenson D M, Murray J L, O'Brian C A, Ioannides C G. Induction of tumor-reactive CTL by C-side chain variants of the CTL epitope HER-2/neu protooncogene (369-377) selected by molecular modeling of the peptide: HLA-A2 complex. J. Immunol. 2002, 169(7):3545-54.
75. Lee C Y, Andersen R O, Cabernard C, Manning L, Tran K D, Lanskey M J, Bashirullah A, Doe C Q. Drosophila Aurora-A kinase inhibits neuroblast self-renewal by regulating aPKC/Numb cortical polarity and spindle orientation. Genes Dev. 2006 Dec. 15; 20(24):3464-74.
76. Miyamoto A, Lau R, Hein P W, Shipley J M, Weinmaster G. Microfibrillar proteins MAGP-1 and MAGP-2 induce Notch1 extracellular domain dissociation and receptor activation. J Biol Chem. 2006 Apr. 14; 281(15):10089-97.

What is claimed is:

1. A method of treating a cancer in a patient, comprising:
immunizing the patient against a peptide derived from a protein selected from the group consisting of Notch1, Notch2, Notch3, and Notch4.

2. The method of claim 1, wherein the peptide is selected from the group consisting of

| | |
|---|---|
| DGVNTYNC, | (SEQ ID NO: 9) |
| RYSRSD, | (SEQ ID NO: 11) |
| LLEASAD, | (SEQ ID NO: 18) |
| LLDEYNLV, | (SEQ ID NO: 21) |
| MPALRPALLWALLALWLCCA, | (SEQ ID NO: 22) |
| NGGVCVDGVNTYNC, | (SEQ ID NO: 25) |
| DGVNTYNCRCPPQWTG, | (SEQ ID NO: 30) |
| RMNDGTTPLI, | (SEQ ID NO: 32) |
| and | |
| LKNGANR. | (SEQ ID NO: 35) |

3. The method of claim 1, wherein the peptide is selected from the group consisting of Notch1₂₇₄₋₂₈₂ (SEQ ID NO:10), Notch1₁₉₃₈₋₁₉₄₃ (SEQ ID NO:11), Notch1₁₉₃₈₋₁₉₄₆ (SEQ ID NO:12), Notch1₁₉₃₈₋₁₉₄₇ (SEQ ID NO:13), Notch1₁₉₄₀₋₁₉₄₈ (SEQ ID NO:14), Notch1₁₉₄₀₋₁₉₄₉ (SEQ ID NO:15), Notch1₁₉₄₄₋₁₉₅₅ (SEQ ID NO:16), Notch1₁₉₄₇₋₁₉₅₅ (SEQ ID NO:17), Notch1₂₁₁₁₋₂₁₂₀ (SEQ ID NO:19), Notch1₂₁₁₂₋₂₁₂₀ (SEQ ID NO:20), Notch1₂₁₁₃₋₂₁₂₀ (SEQ ID NO:21), Notch2₁₋₂₀ (SEQ ID NO:22), Notch2₇₋₁₅ (SEQ ID NO:24), Notch2₂₇₁₋₂₈₅ (SEQ ID NO:26), Notch2₂₇₁₋₂₈₆ (SEQ ID NO:27), Notch2₂₇₇₋₂₈₅ (SEQ ID NO:28), Notch2₂₇₇₋₂₈₆ (SEQ ID NO:29), Notch2₁₉₄₀₋₁₉₄₈ (SEQ ID NO:31), Notch2₁₉₄₀₋₁₉₄₉ (SEQ ID NO:32), Notch21991-2003 (SEQ ID NO:33), Notch2₁₉₉₅₋₂₀₀₃ (SEQ ID NO:34), and Notch2₁₉₉₇₋₂₀₀₃ (SEQ ID NO:35).

4. The method of claim 1, wherein the cancer is selected from the group consisting of T-cell acute lymphoblastic leukemia and lymphoma (T-ALL), breast cancer, ovarian cancer, pancreatic cancer, prostate cancer, liver cancer, stomach cancer, clear-cell renal cell carcinomas, and colon cancer.

5. A composition, comprising:
a peptide derived from a protein selected from the group consisting of Notch1, Notch2, Notch3, and Notch4, and
a pharmaceutically-acceptable carrier.

6. The composition of claim 5, wherein the peptide is selected from the group consisting of DGVNTYNC (SEQ ID NO:9), RYSRSD (SEQ ID NO:11), LLEASAD (SEQ ID NO:18), LLDEYNLV (SEQ ID NO:21), MPALRPALLWALLALWLCCA (SEQ ID NO:22), NGGVCVDGVNTYNC (SEQ ID NO:25), DGVNTYNCRCPPQWTG (SEQ ID NO:30), RMNDGTTPLI (SEQ ID NO:32), and LKNGANR (SEQ ID NO:35).

7. The composition of claim 5, wherein the peptide is selected from the group consisting of wherein the peptide is selected from the group consisting of Notch1₂₇₄₋₂₈₂ (SEQ ID NO:10), Notch1₁₉₃₈₋₁₉₄₃ (SEQ ID NO:11), Notch1₁₉₃₈₋₁₉₄₆ (SEQ ID NO:12), Notch1₁₉₃₈₋₁₉₄₇ (SEQ ID NO:13), Notch1₁₉₄₀₋₁₉₄₈ (SEQ ID NO:14), Notch1₁₉₄₀₋₁₉₄₉ (SEQ ID NO:15), Notch1₁₉₄₄₋₁₉₅₅ (SEQ ID NO:16), Notch1₁₉₄₇₋₁₉₅₅ (SEQ ID NO:17), Notch1₂₁₁₁₋₂₁₂₀ (SEQ ID NO:19), Notch1₂₁₁₂₋₂₁₂₀ (SEQ ID NO:20), Notch1₂₁₁₃₋₂₁₂₀ (SEQ ID NO:21), Notch2₁₋₂₀ (SEQ ID NO:22), Notch2₇₋₁₅ (SEQ ID NO:24), Notch2₂₇₁₋₂₈₅ (SEQ ID NO:26), Notch2₂₇₁₋₂₈₆ (SEQ ID NO:27), Notch2₂₇₇₋₂₈₅ (SEQ ID NO:28), Notch2₂₇₇₋₂₈₆ (SEQ ID NO:29), Notch2₁₉₄₀₋₁₉₄₈ (SEQ ID NO:31), Notch2₁₉₄₀₋₁₉₄₉ (SEQ ID NO:32), Notch2₁₉₉₁₋₂₀₀₃ (SEQ ID NO:33), Notch2₁₉₉₅₋₂₀₀₃ (SEQ ID NO:34), and Notch2₁₉₉₇₋₂₀₀₃ (SEQ ID NO:35).

8. A method of treating a cancer in a patient, comprising:
immunizing the patient against a peptide derived from a protein selected from the group consisting of Numb1, Numb2, Numb3, and Numb4.

9. The method of claim 8, wherein the peptide is selected from the group consisting of

| | |
|---|---|
| LWVSADGL, | (SEQ ID NO: 37) |
| CRDGTTRRWICHCFMAVKD, | (SEQ ID NO: 38) |
| RWICHCFMAVKD, | (SEQ ID NO: 39) |
| RWLEEVSKSVRA, | (SEQ ID NO: 41) |
| and | |
| VDDGRLASADRHTEV. | (SEQ ID NO: 43) |

10. The method of claim 8, wherein the peptide is selected from the group consisting of Numb1₈₇₋₉₅ (SEQ ID NO:36), Numb1₈₈₋₉₅ (SEQ ID NO:37), Numb1₁₃₁₋₁₄₉ (SEQ ID NO:38), Numb1₁₃₈₋₁₄₉ (SEQ ID NO:39), Numb1₁₃₉₋₁₄₇ (SEQ ID NO:40), Numb1₄₄₂₋₄₅₃ (SEQ ID NO:41), Numb1₄₄₃₋₄₅₁ (SEQ ID NO:42), Numb1₅₉₂₋₆₀₆ (SEQ ID NO:43), and Numb1₅₉₄₋₆₀₂ (SEQ ID NO:44).

11. The method of claim 8, wherein the cancer is selected from the group consisting of T-cell acute lymphoblastic leukemia and lymphoma (T-ALL), breast cancer, ovarian cancer, pancreatic cancer, prostate cancer, liver cancer, stomach cancer, clear-cell renal cell carcinomas, and colon cancer.

12. A composition, comprising:
a peptide derived from a protein selected from the group consisting of Numb1, Numb2, Numb3, and Numb4, and
a pharmaceutically-acceptable carrier.

13. The composition of claim 12, wherein the peptide is selected from the group consisting of LWVSADGL (SEQ ID NO:37), CRDGTTRRWICHCFMAVKD (SEQ ID NO:38), RWICHCFMAVKD (SEQ ID NO:39), RWLEEVSKSVRA (SEQ ID NO:41), and VDDGRLASADRHTEV (SEQ ID NO:43).

14. The composition of claim 12, wherein the peptide is selected from the group consisting of wherein the peptide is selected from the group consisting of Numb1₈₇₋₉₅ (SEQ ID NO:36), Numb1₈₈₋₉₅ (SEQ ID NO:37), Numb1₁₃₁₋₁₄₉ (SEQ ID NO:38), Numb1₁₃₈₋₁₄₉ (SEQ ID NO:39), Numb1₁₃₉₋₁₄₇ (SEQ ID NO:40), Numb1₄₄₂₋₄₅₃ (SEQ ID NO:41), Numb1₄₄₃₋₄₅₁ (SEQ ID NO:42), Numb1₅₉₂₋₆₀₆ (SEQ ID NO:43), and Numb1₅₉₄₋₆₀₂ (SEQ ID NO:44).

15. A method of treating a cancer in a patient, comprising:
administering to the patient a composition comprising an antibody against a peptide derived from a protein selected from the group consisting of Notch1, Notch2, Notch3, Notch4, Numb1, Numb2, Numb3, and Numb4.

16. The method of claim 15, wherein the peptide is selected from the group consisting of

| | |
|---|---|
| DGVNTYNC, | (SEQ ID NO: 9) |
| RYSRSD, | (SEQ ID NO: 11) |
| LLEASAD, | (SEQ ID NO: 18) |
| LLDEYNLV, | (SEQ ID NO: 21) |
| MPALRPALLWALLALWLCCA, | (SEQ ID NO: 22) |
| NGGVCVDGVNTYNC, | (SEQ ID NO: 25) |
| DGVNTYNCRCPPQWTG, | (SEQ ID NO: 30) |
| RMNDGTTPLI, | (SEQ ID NO: 32) |
| LKNGANR, | (SEQ ID NO: 35) |
| LWVSADGL, | (SEQ ID NO: 37) |
| CRDGTTRRWICHCFMAVKD, | (SEQ ID NO: 38) |
| RWICHCFMAVKD, | (SEQ ID NO: 39) |
| RWLEEVSKSVRA, | (SEQ ID NO: 41) |
| and | |
| VDDGRLASADRHTEV. | (SEQ ID NO: 43) |

17. The method of claim 15, wherein the peptide is selected from the group consisting of Notch1₂₇₄₋₂₈₂ (SEQ ID NO:10), Notch1₁₉₃₈₋₁₉₄₃ (SEQ ID NO:11), Notch1₁₉₃₈₋₁₉₄₆ (SEQ ID NO:12), Notch1₁₉₃₈₋₁₉₄₇ (SEQ ID NO:13), Notch1₁₉₄₀₋₁₉₄₈ (SEQ ID NO:14), Notch1₁₉₄₀₋₁₉₄₉ (SEQ ID NO:15), Notch1₁₉₄₄₋₁₉₅₅ (SEQ ID NO:16), Notch1₁₉₄₇₋₁₉₅₅ (SEQ ID NO:17), Notch1₂₁₁₁₋₂₁₂₀ (SEQ ID NO:19), Notch1₂₁₁₂₋₂₁₂₀ (SEQ ID NO:20), Notch1₂₁₁₃₋₂₁₂₀ (SEQ ID NO:21), Notch2₁₋₂₀ (SEQ ID NO:22), Notch2₇₋₁₅ (SEQ ID NO:24), Notch2₂₇₁₋₂₈₅ (SEQ ID NO:26), Notch2₂₇₁₋₂₈₆ (SEQ ID NO:27), Notch2₂₇₇₋₂₈₅ (SEQ ID NO:28), Notch2₂₇₇₋₂₈₆ (SEQ ID NO:29), Notch2₁₉₄₀₋₁₉₄₈ (SEQ ID NO:31), Notch2₁₉₄₀₋₁₉₄₉ (SEQ ID NO:32), Notch2₁₉₉₁₋₂₀₀₃ (SEQ ID NO:33), Notch2₁₉₉₅₋₂₀₀₃ (SEQ ID NO:34), Notch2₁₉₉₇₋₂₀₀₃ (SEQ ID NO:35), Numb1₄₄₃₋₄₅₁ (SEQ ID NO:36), Numb1₈₈₋₉₅ (SEQ ID NO:37), Numb1₁₃₁₋₁₄₉ (SEQ ID NO:38), Numb1₁₃₈₋₁₄₉ (SEQ ID NO:39), Numb1₁₃₉₋₁₄₇ (SEQ ID NO:40), Numb1₄₄₂₋₄₅₃ (SEQ ID NO:41), Numb1₄₄₃₋₄₅₁ (SEQ ID NO:42), Numb1₅₉₂₋₆₀₆ (SEQ ID NO:43), and Numb1₅₉₄₋₆₀₂ (SEQ ID NO:44).

18. The method of claim 15, wherein the cancer is selected from the group consisting of T-cell acute lymphoblastic leukemia and lymphoma (T-ALL), breast cancer, ovarian cancer, pancreatic cancer, prostate cancer, liver cancer, stomach cancer, clear-cell renal cell carcinomas, and colon cancer.

19. The method of claim 15, wherein the composition further comprises a therapeutic molecule selected from the group consisting of anti-cancer drugs and radioisotopes.

20. The method of claim 19, wherein the therapeutic molecule is covalently linked to a constant region of a heavy chain of the antibody.


SEQUENCE LISTING (44 sequences)

SEQ ID NO: 1 - protein, 2556 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQAGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 2 - protein, 2471 aa, Homo sapiens
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 3 - protein, 2321 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPVAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 4 - protein, 1999 aa, Homo sapiens
MQPPSLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPGCEVNPDNCVSHQCQNGGTCQDGLDTYTCLCPETWTGWDCSEDVDECEAQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 5 - protein, 651 aa, Homo sapiens
MNKLRQSFRRKKDVYVPEASRPHQWQTDEEGVRTGKCSFPVKYLGHVEVDESRGMHICEDAVKRLKAERKFFKGFFGKTGKKAVKAVLWVSADGLRVVDEKTKDLIVDQTIEKVSFCAPDRNFDRAFSYICRDGTTRRWICHCFMAVKDTGERLSHAVGCAFAACLERKQKREKECGVTATFDASRTTFTREGSFRVTTATEQAEREEIMKQMQDAKKAETDKIVVGSSVAPGNTAPSPSSPTSPTSDATTSLEMNNPHAIPRRHAPIEQLARQGSFRGFPALSQKMSPFKRQLSLRINELPSTMQRKTDFPIKNAVPEVEGEAESISSLCSQITNAFSTPEDPFSSAPMTKPVTVVAPQSPTFQANGTDSAFHVLAKPAHTALAPVAMPVRETNPWAHAPDAANKEIAATCSGTEWGQSSGAASPGLFQAGHRRTPSEADRWLEEVSKSVRAQQPQASAAPLQPVLQPPPPTAISQPASPFQGNAFLTSQPVPVGVVPALQPAFVPAQSYPVANGMPYPAPNVPVVGITPSQMVANVFGTAGHPQAAHPHQSPSLVRQQTFPHYEASSATTSPFFKPPAQHLNGSAAFNGVDDGRLASADRHTEVPTGTCPVDPFEAQWAALENKSKQRTNPSPTNPFSSDLQKTFEIEL

SEQ ID NO: 6 - protein, 603 aa, Homo sapiens
MNKLRQSFRRKKDVYVPEASRPHQWQTDEEGVRTGKCSFPVKYLGHVEVDESRGMHICEDAVKRLKAERKFFKGFFGKTGKKAVKAVLWVSADGLRVVDEKTKDLIVDQTIEKVSFCAPDRNFDRAFSYICRDGTTRRWICHCFMAVKDTGERLSHAVGCAFAACLERKQKREKECGVTATFDASRTTFTREGSFRVTTATEQAEREEIMKQMQDAKKAETDKIVVGSSVAPGNTAPSPSSPTSPTSDATTSLEMNNPHAIPRRHAPIEQLARQGSFRGFPALSQKMSPFKRQLSLRINELPSTMQRKTDFPIKNAVPEVEGEAESISSLCSQITNAFSTPEDPFSSAPMTKPVTVVAPQSPTFQGTEWGQSSGAASPGLFQAGHRRTPSEADRWLEEVSKSVRAQQPQASAAPLQPVLQPPPPTAISQPASPFQGNAFLTSQPVPVGVVPALQPAFVPAQSYPVANGMPYPAPNVPVVGITPSQMVANVFGTAGHPQAAHPHQSPSLVRQQTFPHYEASSATTSPFFKPPAQHLNGSAAFNGVDDGRLASADRHTEVPTGTCPVDPFEAQWAALENKSKQRTNPSPTNPFSSDLQKTFEIEL

SEQ ID NO: 7 - protein, 640 aa, Homo sapiens
MNKLRQSFRRKKDVYVPEASRPHQWQTDEEGVRTGKCSFPVKYLGHVEVDESRGMHICEDAVKRLKATGKKAVKAVLWVSADGLRVVDEKTKDLIVDQTIEKVSFCAPDRNFDRAFSYICRDGTTRRWICHCFMAVKDTGERLSHAVGCAFAACLERKQKREKECGVTATFDASRTTFTREGSFRVTTATEQAEREEIMKQMQDAKKAETDKIVVGSSVAPGNTAPSPSSPTSPTSDATTSLEMNNPHAIPRRHAPIEQLARQGSFRGFPALSQKMSPFKRQLSLRINELPSTMQRKTDFPIKNAVPEVEGEAESISSLCSQITNAFSTPEDPFSSAPMTKPVTVVAPQSPTFQANGTDSAFHVLAKPAHTALAPVAMPVRETNPWAHAPDAANKEIAATCSGTEWGQSSGAASPGLFQAGHRRTPSEADRWLEEVSKSVRAQQPQASAAPLQPVLQPPPPTAISQPASPFQGNAFLTSQPVPVGVVPALQPAFVPAQSYPVANGMPYPAPNVPVVGITPSQMVANVFGTAGHPQAAHPHQSPSLVRQQTFPHYEASSATTSPFFKPPAQHLNGSAAFNGVDDGRLASADRHTEVPTGTCPVDPFEAQWAALENKSKQRTNPSPTNPFSSDLQKTFEIEL

SEQ ID NO: 8 - protein, 592 aa, Homo sapiens
MNKLRQSFRRKKDVYVPEASRPHQWQTDEEGVRTGKCSFPVKYLGHVEVDESRGMHICEDAVKRLKATGKKAVKAVLWVSADGLRVVDEKTKDLIVDQTIEKVSFCAPDRNFDRAFSYICRDGTTRRWICHCFMAVKDTGERLSHAVGCAFAACLERKQKREKECGVTATFDASRTTFTREGSFRVTTATEQAEREEIMKQMQDAKKAETDKIVVGSSVAPGNTAPSPSSPTSPTSDATTSLEMNNPHAIPRRHAPIEQLARQGSFRGFPALSQKMSPFKRQLSLRINELPSTMQRKTDFPIKNAVPEVEGEAESISSLCSQITNAFSTPEDPFSSAPMTKPVTVVAPQSPTFQGTEWGQSSGAASPGLFQAGHRRTPSEADRWLEEVSKSVRAQQPQASAAPLQPVLQPPPPTAISQPASPFQGNAFLTSQPVPVGVVPALQPAFVPAQSYPVANGMPYPAPNVPVVGITPSQMVANVFGTAGHPQAAHPHQSPSLVRQQTFPHYEASSATTSPFFKPPAQHLNGSAAFNGVDDGRLASADRHTEVPTGTCPVDPFEAQWAALENKSKQRTNPSPTNPFSSDLQKTFEIEL

SEQ ID NO: 9 - protein, 8 aa, Homo sapiens
DGVNTYNC

SEQ ID NO: 10 - protein, 9 aa, Homo sapiens
DGVNTYNCR

SEQ ID NO: 11 - protein, 6 aa, Homo sapiens
RYSRSD

SEQ ID NO: 12 - protein, 9 aa, Homo sapiens
RYSRSDAAK

SEQ ID NO: 13 - protein, 10 aa, Homo sapiens
RYSRSDAAKR

SEQ ID NO: 14 - protein, 9 aa, Homo sapiens
SRSDAAKRL

SEQ ID NO: 15 - protein, 10 aa, Homo sapiens
SRSDAAKRLL

SEQ ID NO: 16 - protein, 12 aa, Homo sapiens
AAKRLLEASADA

SEQ ID NO: 17 - protein, 9 aa, Homo sapiens
RLLEASADA

SEQ ID NO: 18 - protein, 7 aa, Homo sapiens
LLEASAD

SEQ ID NO: 19 - protein, 10 aa, Homo sapiens
VRLLDEYNLV

SEQ ID NO: 20 - protein, 9 aa, Homo sapiens
RLLDEYNLV

SEQ ID NO: 21 - protein, 8 aa, Homo sapiens
LLDEYNLV

SEQ ID NO: 22 - protein, 20 aa, Homo sapiens
MPALRPALLWALLALWLCCA

SEQ ID NO: 23 - protein, 20 aa, Homo sapiens
MPALRPALLWALLALWLCCA

SEQ ID NO: 24 - protein, 9 aa, Homo sapiens
ALLWALLAL

SEQ ID NO: 25 - protein, 14 aa, Homo sapiens
NGGVCVDGVNTYNC

SEQ ID NO: 26 - protein, 15 aa, Homo sapiens
NGGVCVDGVNTYNCR

SEQ ID NO: 27 - protein, 16 aa, Homo sapiens
NGGVCVDGVNTYNCRC

SEQ ID NO: 28 - protein, 9 aa, Homo sapiens
DGVNTYNCR

SEQ ID NO: 29 - protein, 10 aa, Homo sapiens
DGVNTYNCRC

SEQ ID NO: 30 - protein, 16 aa, Homo sapiens
DGVNTYNCRCPPQWTG

SEQ ID NO: 31 - protein, 9 aa, Homo sapiens
RMNDGTTPL

SEQ ID NO: 32 - protein, 10 aa, Homo sapiens
RMNDGTTPLI

SEQ ID NO: 33 - protein, 13 aa, Homo sapiens
EATLLLLKNGANR

SEQ ID NO: 34 - protein, 9 aa, Homo sapiens
LLLKNGANR

SEQ ID NO: 35 - protein, 7 aa, Homo sapiens
LKNGANR

SEQ ID NO: 36 - protein, 9 aa, Homo sapiens
VLWVSADGL

SEQ ID NO: 37 - protein, 8 aa, Homo sapiens
LWVSADGL

SEQ ID NO: 38 - protein, 19 aa, Homo sapiens
CRDGTTRRWICHCFMAVKD

SEQ ID NO: 39 - protein, 12 aa, Homo sapiens
RWICHCFMAVKD

SEQ ID NO: 40 - protein, 9 aa, Homo sapiens
WICHCFMAV

SEQ ID NO: 41 - protein, 12 aa, Homo sapiens
RWLEEVSKSVRA

SEQ ID NO: 42 - protein, 9 aa, Homo sapiens
WLEEVSKSV

SEQ ID NO: 43 - protein, 15 aa, Homo sapiens
VDDGRLASADRHTEV

SEQ ID NO: 44 - protein, 9 aa, Homo sapiens
DGRLASADR